(19) United States
(12) Patent Application Publication
(10) Pub. No.: US 2024/0210548 A1
(43) Pub. Date: Jun. 27, 2024

(54) METHODS AND APPARATUS TO MONITOR DISTANCES TRAVELED BY SUBJECTS DURING SIX-MINUTE WALK TESTS

(71) Applicant: Abbott Laboratories, Abbott Park, IL (US)

(72) Inventors: David J. Billick, Wilton, CT (US); Donovan D. Remaley, Fairfield, CT (US)

(21) Appl. No.: 18/393,112

(22) Filed: Dec. 21, 2023

Related U.S. Application Data
(60) Provisional application No. 63/476,571, filed on Dec. 21, 2022.

(51) Int. Cl. G01S 11/06; A61B 5/02; H04B 17/318

(52) U.S. Cl. CPC G01S 11/06; A61B 5/02; H04B 17/318

(57) ABSTRACT

Methods and apparatus to monitor distances traveled by subjects during six-minute walk tests are disclosed. An example system includes a mobile beacon to be carried by a subject during a six-minute walk test, interface circuitry, machine readable instructions, and programmable circuitry to at least one of instantiate or execute the machine readable instructions to determine a distance traveled by the subject during the six-minute walk test based on a strength of signals received from the mobile beacon.

### RELATED APPLICATION

This patent claims the benefit of U.S. Provisional Patent Application No. 63/476,571, which was filed on Dec. 21, 2022. U.S. Provisional Patent Application No. 63/476,571 is hereby incorporated herein by reference in its entirety. Priority to U.S. Provisional Patent Application No. 63/476,571 is hereby claimed.

### FIELD OF THE DISCLOSURE

This disclosure relates generally to testing cardiopulmonary health and, more particularly, to methods and apparatus to monitor distances traveled by subjects during six-minute walk tests.

### BACKGROUND

During a six-minute walk test, a subject's performance is measured by the distance they walk over a six-minute span. The six-minute walk test is administered in clinical settings with healthcare provider supervision. The six-minute walk test is often the clinical test of choice to assess functional exercise capacity in subjects of all ages that deal with, or have dealt with, cardiopulmonary issues, arthritis, stroke, fibromyalgia, geriatrics, Multiple Sclerosis, Parkinson's Disease, and/or other health related issues.

### BRIEF DESCRIPTION OF THE DRAWINGS

FIG. 1A illustrates an example six-minute walk test system in accordance with teachings disclosed herein.

FIG. 1B illustrates another example six-minute walk test system in accordance with teachings disclosed herein.

FIG. 1C illustrates another example six-minute walk test system in accordance with teachings disclosed herein.

FIG. 1D illustrates another example six-minute walk test system in accordance with teachings disclosed herein.

FIG. 2 is a block diagram of an example mobile device of the six-minute walk test system of FIGS. 1A-1D.

FIG. 3 is a block diagram of an example external device of the six-minute walk test system of FIGS. 1A-1D.

FIGS. 4A-4C are graphical depictions of Bluetooth beacon signal processing performed by the example external device of FIG. 3.

FIG. 5 is a flowchart representative of example machine readable instructions and/or example operations that may be executed by example programmable circuitry to implement the example mobile device of FIG. 2.

FIG. 6 is a flowchart representative of example machine readable instructions and/or example operations that may be executed by example programmable circuitry to implement the example external device of FIG. 3.

FIG. 7 is a block diagram of an example processing platform including programmable circuitry structured to execute the example machine readable instructions and/or the example operations of FIG. 5 to implement the example mobile device of FIG. 2.

FIG. 8 is a block diagram of an example processing platform including programmable circuitry structured to execute the example machine readable instructions and/or the example operations of FIG. 6 to implement the example external device of FIG. 3.

FIG. 9 is a block diagram of an example implementation of the programmable circuitry of FIGS. 7 and/or 8.

FIG. 10 is a block diagram of another example implementation of the programmable circuitry of FIGS. 7 and/or 8.

FIG. 11 is a block diagram of an example software distribution platform (e.g., one or more servers) to distribute software (e.g., software corresponding to the example machine readable instructions of FIGS. 5-6) to client devices associated with end users and/or consumers (e.g., for license, sale, and/or use), retailers (e.g., for sale, re-sale, license, and/or sub-license), and/or original equipment manufacturers (OEMs) (e.g., for inclusion in products to be distributed to, for example, retailers and/or to other end users such as direct buy customers).

In general, the same reference numbers will be used throughout the drawing(s) and accompanying written description to refer to the same or like parts. The figures are not to scale.

### DETAILED DESCRIPTION

Fitness levels including cardiopulmonary health of patients can be assessed through different types of testing including, for example, the six-minute walk test. The American Thoracic Society describes the six-minute walk test as a measure of functional status or fitness. The distance traveled by the subject during the test and health parameters, such as for example, blood pressure, heart rate, and oxygen saturation, are measured before and after the test to provide insight into the subject's health status. The results may be indicative of whether more sophisticated measures of the subject's heart and lung function should be taken. For instance, the results of the test can be compared to previous results for the subject and/or a normal result range given the subject's age, height, weight, and/or gender.

To administer the test, the subject travels to a clinical setting where a test administrator has a walkable course set up. For instance, the course can include two cones set up between 20 and 40 feet apart. During the test, the subject travels back and forth between the cones, and the test administrator counts the number of trips (e.g., from one end cone to another) that the subject completes. When the six-minute period is complete, the subject stops where they are, and the test administrator measures the distance traveled by the subject during the uncompleted trip via a tape measure or some other distance measuring tool. In turn, the test administrator multiplies the number of trips by the distance between the cones (e.g., 20 feet, 40 feet, etc.) and adds the distance traveled during the last, uncompleted trip to obtain the total distance that the subject walked during the six-minute period. Accordingly, the test is fairly simple and easy to administer.

Example methods and apparatus to monitor six-minute walk tests in a non-clinical setting are disclosed herein. For instance, disclosed examples enable a subject to perform the six-minute walk test at their residence, on vacation, and/or at any other suitable location outside the clinical setting without the assistance of a clinician or any other individual. The results of the six-minute walk tests administered in a non-clinical setting are transmissible to a remote facility and/or other networked device. Thus, the subject's health care provider can access the test results to determine whether the patient is performing as expected, assess the test results, and/or identify when additional testing should be conducted. As a result, examples disclosed herein enable the health of a subject to be monitored without any inconvenience for the subject to travel to and from a clinic, which can help save the subject and/or caretaker time, travel expenses, and/or health care fees. Examples disclosed herein also reduce the time needed by clinicians for administering the six-minute walk test.

An example six-minute walk test system disclosed herein includes a mobile beacon (e.g., mobile Bluetooth beacon) to be carried by the subject during the test. For example, the mobile beacon can be attached to a lanyard, a wristband, an arm band, a headband, a clip, or any other object that the subject can comfortably carry and/or wear while walking. Advantageously, the relatively small size of the mobile beacon helps minimize any added weight carried by the subject during the test. In examples disclosed herein, the six-minute walk test system includes a mobile device (e.g., a tablet, a smartphone, etc.) on which an application can be downloaded or otherwise installed and utilized to provide instructions for the test as well as present test results.

Furthermore, in some examples, the six-minute walk test system disclosed herein includes end-marking objects to be positioned at opposite end points (e.g., pivot points) of the course that the subject traverses during the test. In some examples, the end-marking objects are a simple marker (e.g., cones, tape, ends of stick or string, etc.) indicative of an end of the track and, thus, a turnaround point for the subject. In some examples, the subject utilizes household items (e.g., furniture, paintings, lamps, etc.) as the end-marking objects.

In some examples, the end-marking objects include two stationary beacons (e.g., Bluetooth beacons). In some examples, the beacons are relatively small in size and can be attached to a cone or some other object to help the subject identify the course while walking between the end points. In some examples, the stationary beacons are connected via a string or some other material that extends the distance of the course (e.g., 20 feet) to help the subject accurately measure the course.

In some examples, the end-marking objects include proximity or sonar sensors. In such examples, as the patient approaches a proximity or sonar sensor at an end of the track, an ultrasonic sound wave will reflect off the patient and return to the sensor. Further, a location can be calculated by measuring the time the waves take to reach the sensor. In some examples, the respective proximity or sonar sensors at the end of the track detect a location of the subject at a single point at the respective ends of the track (e.g., are triggered when the subject arrives at the end of the track). In some other examples, the proximity or sonar sensors detect a range of locations along the track. In such examples, the respective proximity or sonar sensors at the end of the track can detect a position of the subject at various locations between the ends of the track based on an amount of time that the ultrasonic wave takes to return to the sensor. In some examples, a plurality of the proximity or sonar sensors are spaced apart along the track to detect a position of the subject as they travel from one end of the track to another.

In some examples, the end-marking objects include infrared sensors. In such examples, as the subject traverses the track, the infrared sensors monitor infrared radiation in the electromagnetic spectrum (e.g., light reflected and/or heat emitted by the subject). When the infrared radiation satisfies (e.g., is greater than, is greater than or equal to) a threshold, the subject has reached the end of the track. Similar to the proximity or sonar sensors, a plurality of the infrared sensors can be spaced apart along the track to detect a position of the subject as they travel from one end of the track to another.

In some examples, the end-marking objects include optical sensors (e.g., cameras) to track patient movements. In such examples, images captured by the optical sensors can be processed (e.g., via green-screen and/or machine learning) to determine a patient's distance and movement relative to the six-minute walk test track. Additionally, the images captured by the optical sensors can be analyzed to verify that the subject complied with the parameters of the test (e.g., verify that the subject traversed the full distance of the track during each traversal). In some examples, the optical sensors are triggered to capture image data when a specific behavior is flagged, such as when the subject reaches an end of the track or when the subject leaves the track. In some other examples, the optical sensors retain all frames captured during the six-minute walk test for analysis.

In some examples, the end-marking objects include Wi-Fi or radio receivers. In such examples, the subject carries a Wi-Fi or radio beacon that emits radio signals that are detected by the end marking Wi-Fi or radio receivers. In such examples, distance between the subject and one or both of the end-marking objects is determined based on (i) a strength of the signal received by the end-marking objects (e.g., received signal strength indication (RSSI)) or (ii) a time that it takes for the signal emitted by the beacon to be detected by the end-marking Wi-Fi or radio receiver (e.g., time of flight (ToF)). In some other examples, the end-marking objects include Wi-Fi or radio transmitters and the subject carries a Wi-Fi or radio receiver. In such examples, another Wi-Fi or radio transmitter is positioned outside of the track to enable triangulation or trilateration to be utilized to determine the position of the subject.

In some examples, the end-marking objects and/or circuitry carried by the subject (e.g., the mobile beacon, the Wi-Fi or radio transmitter or receiver, vital sign sensors carried by the subject during the test, etc.) include smart sensor circuitry. In such examples, ones of the smart sensor circuitry are part of a micro-mesh network that enables the end-marking objects and/or circuitry carried by the subject to communicate and coordinate operation. Accordingly, the smart sensor circuitry can include first smart sensor circuitry that the subject carries as the subject moves during the test and at least one second smart sensor circuitry that is stationary and in communication with the first smart sensor circuitry.

The subject can begin the test at a first end-marking object (e.g., a first cone, a first stationary beacon, a first sensor, etc.), walk to a second end-marking object (e.g., a second cone, a second stationary beacon, a second sensor, etc.) a distance (e.g., approximately 20-40 feet) away from the first end-marking object and turn around and walk back to the first end-marking object. The subject repeats this process until the end of the six-minute period while taking breaks to rest if needed. In some examples, the six-minute walk test system does not include the end-marking objects and the subject identifies the locations at which they are to turn around in another manner, such as for example, from memory of the general location, from instruction by another party, from counting paces, etc.

When the subject is preparing for the test, the mobile device can provide instructions regarding the equipment set up, pre-test rest, and pre-test measurements. For example, the mobile device can help the subject identify the equipment that should be present and instruct the subject on how to measure the walking track. After the track is ready, the mobile device instructs the patient to rest for a certain period (e.g., 10 minutes) before measuring their vital signs (e.g., heart rate, blood pressure, blood oxygen saturation). Once the measured vital signs are recorded on the mobile device, the mobile device can provide instructions, or reminders, on how to complete the six-minute walk test. In some examples, when the subject is ready to begin the test, the mobile device instructs the subject to place the mobile device in a location at an end of the walking track. For example, one of the end-marking objects can be positioned between the mobile device and the course. In some other examples, when the subject is ready to begin the test, the mobile device instructs the subject to place the mobile device in another location adjacent to the walking track (e.g., within a certain distance of the track between parallel planes defined by the end-marking objects perpendicular to the walking track). The subject can initiate a timer that counts down to the start of the test before or after positioning the mobile device and walk to the first end of the track where the subject will begin the test. In turn, the mobile device can provide an audible and/or visual indication of a start of the test at which point the subject will begin to walk back and forth between the ends of the track.

During the test, the mobile device receives signals transmitted by the mobile beacon carried by the subject. In some examples, the mobile device identifies the signal that originated from the mobile beacon based on an identifier carried by the signal transmitted by the mobile beacon. In some examples, when the six-minute walk test system utilizes stationary beacons as the end-marking objects associated with the track, the mobile device identifies an expected signal strength range of the signals transmitted by the mobile beacon during the test based on the signals received from the stationary beacons. Specifically, a first signal strength from a first stationary beacon (e.g., the stationary beacon closer to the mobile device) can define a maximum expected signal strength, and a second signal strength from a second stationary beacon (e.g., the stationary beacon further from the mobile device) can define a minimum expected signal strength. Other devices (e.g., kitchen appliances, a television, a speaker, etc.) near the course may also emit signals that can interfere with the signals from the mobile beacon. However, the signals from the stationary beacons establish a range of signal strengths of interest, which enables the mobile device to ignore signals outside of the expected signal strength range and, thus, adapt to the testing environment. As such, the stationary beacons help the six-minute walk test system minimize or otherwise reduce signal processing performed by the mobile device because the mobile device can remove or ignore signals from the other devices from its signal processing.

In some examples, the mobile device instructs the subject to utilize the stationary beacons as the end-marking objects in response to detecting possible interference in advance of the test. For example, the mobile device can monitor for external signals when providing the instructions regarding the equipment set up, pre-test rest, and pre-test measurements. In some examples, the mobile device determines that the stationary beacon end markers are to be set up at the ends of the track in response to (i) detecting an external signal strength within a threshold range and/or (ii) detecting a quantity of external signals that satisfies a threshold quantity. Further, the mobile device can indicate whether the stationary beacons are to be utilized and enable the subject to set up the stationary beacons at the ends of the track in advance of the subject beginning their rest period.

In some examples, throughout the test, the mobile device measures a strength (e.g., an attenuation) of the signal from the mobile beacon and records the measured strength with a time stamp. In some examples, because Bluetooth beacons are configured for low battery consumption, the mobile beacon transmits signals at a first frequency (e.g., once per second). To enable consistent tracking of the mobile beacon, the mobile device pings the mobile beacon at a second frequency (e.g., twice per second), which causes the mobile beacon to transmit signals more frequently thereby enabling subject position information to be determined with increased accuracy. When the mobile device determines that the signal associated with the mobile beacon is not being received or is being received with approximately the same signal strength for a certain period (e.g., 5 seconds), the mobile device can prompt the subject to ensure that they are carrying the mobile beacon and/or that the mobile beacon has battery power and is powered on. In some examples, the mobile device receives data from an end-marking device (e.g., end marker circuitry).

When the six-minute period expires, the mobile device transmits the raw signal strength data to an external device (e.g., a computer, a server, etc.). The external device normalizes the signal data and removes any outliers. For example, the external device can perform 5-point averaging for each signal strength data point to remove jitter and enable the strength to be more consistently indicative of the subject's travel over time.

In some examples, to identify trips that the subject completed between the ends of the track, the external device identifies changes in a slope of the signal strength. For example, the external device can determine that the signal strength is initially decreasing over time as the subject travels from the closer end of the track to the further end of the track. Further, the external device can determine that the subject reached the further end of the track and turned around when a threshold increase in signal strength (e.g., an increase of 5 decibels (dB) when the signal strength had been decreasing over time) is encountered. Similarly, the external device can determine that the subject reached the closer end of the track and turned around when a threshold decrease in signal strength (e.g., a decrease of 5 decibels (dB) when the signal strength had been increasing over time) is encountered. Defining the thresholds indicative of the subject turning around at the end of the track enables the external device to avoid attributing the subject turning around at an end of the course to smaller strength changes that result from random signal variation.

In some examples, the external device modifies the threshold increase and the threshold decrease (e.g., threshold signal strength slope changes) associated with the subject turning around at an end of the track (e.g., a turnaround determination) based on an estimated speed of the subject. For example, the external device can estimate the speed at which the subject was moving during the test based on a slope of the signal strength over time, which corresponds to the change in the signal strength that the mobile device received over time. More particularly, the external device determines that the subject was moving at a slower pace when there is a relatively lower slope. When the external device determines that the subject was walking relatively slower, the external device can increase the threshold change in signal strength for the turnaround determination (e.g., from 10% of the difference between a maximum and minimum signal strength in the data to 40% of the difference). Specifically, because there is often natural variance in the signal strength that the mobile device receives from the mobile beacon even without movement, and because such natural variance is more prevalent when the subject travels at a slower pace, the external device adjusts the threshold increase and the threshold decrease associated with the turnaround determination to prevent the natural variance in the signal strength from resulting in an inaccurately flagged (e.g., false positive) turnaround.

In some examples, in response to identifying the threshold increase or decrease, the external device can identify a local minimum or maximum signal strength encountered at a time before the threshold increase or decrease and associate the data point with the subject being at an end of the track. In such examples, the external device associates each of the identified local minimums and maximums with a completed trip between the ends of the track (e.g., from one end of the track to another). The external device determines a quantity of traversals that the subject completed between the stationary beacons based the number of identified local minimums and maximums or signal strength changes that satisfy the turning point thresholds. Further, the external device multiplies the quantity of traversals by the predetermined distance associated with each lap (e.g., 20 feet). The distance associated with each lap is predetermined because the encountered signal strength range is dependent on the testing environment and, thus, a certain signal strength range is not consistently attributable to a certain distance.

In some examples, when the six-minute period for the test expires after the subject has started but not completed a new or subsequent trip, the mobile device determines a distance that the subject traveled during the partially completed trip. In some examples, the external device identifies an incomplete trip between the ends of the track in response to an identified local minimum or maximum not being within a threshold range of an average local minimum or maximum for the test. For example, the external device can determine the subject turned around before reaching the further end of the track in response to an identified local minimum being 15 dB greater than the average local minimum for the test. Further, the external device can determine the distance associated with any incomplete trips based on a ratio between the change in signal strength associated with a full trip and a change in the signal strength encountered during the partially completed trip. The external device can multiply the predetermined distance of the course by the number of completed trips and add the distance(s) of any partially completed trips to calculate the total distance traveled by the subject during the six-minute walk test. The external device transmits the total distance traveled during the six-minute walk test to the mobile device.

In some examples, the external device determines a confidence score for the test results based on a standard deviation of the local minimums, a standard deviation of the local maximums, and/or a signal strength range associated with each trip. For example, when the signal strength range associated with each trip (e.g., a difference between the local minimums and the local maximums) does not satisfy (e.g., is less than, is less than or equal to) a predetermined threshold (e.g., 15 dB, 20 dB, etc.), the external device can assign a lower confidence score to the test. In some examples, when the confidence score for the test does not satisfy (e.g., is less than, is less than or equal to) a threshold, the external device can determine an action for the subject to take to improve a reliability of the test. For example, the external device can determine that the subject may need one or more new beacons or that the subject may need to perform the test in a different environment with less signal interference. In such examples, the external device can transmit a signal indicative of the determined action to the mobile device.

In some examples, the mobile device is positioned a certain distance from the walking track in an area between the two ends of the track as opposed to at an end of the track. In such examples, a local maximum in the signal strength corresponds to the subject passing the mobile device (e.g., being at a location within the track closest to the mobile device). Further, the local minimums in the signal strength correspond to the subject reaching an end of the track. In some examples, when the mobile device is not aligned with a mid-point of the track between the ends of the track, the local minimums have different amplitudes as the ends of the track are separated from the mobile device by different distances. The external device can similarly process the changes in slope and the local minimums and maximums in the signal strength data to determine the quantity of traversals that the subject made between the ends of the track. In some examples, the mobile device prompts the user to indicate whether the mobile device is positioned at an end of the track, a middle or midpoint of the track between the two ends of the track, or offset from a middle of the track. In such examples, the external device determines subject positions to attribute to certain data points based on the indicated test setup. For example, when the subject indicates that the mobile device is at an end of the track, the external device can determine a lap (e.g., a back and forth traversal) between the ends of the track was completed in response to identifying a minimum signal strength, a maximum signal strength, and another minimum signal strength or a maximum signal strength, a minimum signal strength, and another maximum signal strength. Further, when the subject indicates that the mobile device is offset from a middle portion of the track (e.g., between parallel planes defined by the ends of the track), the external device can determine a lap between the ends of the track was completed in response to identifying a first minimum signal strength, a maximum signal strength, a second minimum signal strength, the maximum signal strength, and the first minimum signal strength.

In examples throughout this disclosure, the mobile device can perform one or more or all the calculations, signal processing, and/or other functions of the external device.

In response to completion of the test, the mobile device can instruct the subject to obtain measurements of post-test vital signs. In some examples, the mobile device prompts the subject to enter a quantity of periodic rests that the subject took during the test. For example, if the subject stopped four different times during the six-minute period to rest, they would indicate this quantity via the mobile device. The mobile device can transmit the indicated quantity of periodic rests to the external device. In some examples, the external device determines a quantity of periodic rests that the subject took based on the signal strength data. In some examples, when the indicated quantity of periodic rests and/or the determined quantity of periodic rests satisfies (e.g., is greater than, is greater than or equal to) a threshold, the external device infers that the subject is a relatively slow walker and adjusts (e.g., increases) the threshold change in signal strength associated with a turnaround, as discussed above.

In some examples, the external device compares the indicated quantity of periodic rests to the determined quantity of periodic rests. When the external device determines that the indicated quantity of periodic rests differs from the determined quantity of periodic rests by at least a threshold amount (e.g., 1, 2, etc.), the external device can cause the confidence score associated with the particular test to be reduced. In some examples, when the confidence score does not satisfy (e.g., is less than, is less than or equal to) a confidence threshold, the external device transmits a low confidence score indication to the mobile device and/or to another device associated with a healthcare provider for the subject. For example, the low confidence score indication can include a recommendation that the test be performed in a different setting, such as a clinical setting.

In some examples, the mobile device presents the total distance traveled to the subject along with the pre-test and/or post-test vital signs. In some examples, the mobile device displays the total distance traveled and/or the vital signs in a format that compares the results of the instant test with previous tests and/or goals. In some examples, the mobile device prompts the user to take any action suggested by the external device for improved test reliability. For example, the mobile device can prompt the user to utilize a different location for a subsequent test. Furthermore, the mobile device and/or the external device can store and/or transmit the test results for further analysis by a health care provider associated with the subject. Accordingly, the health care provider can determine whether the health of the subject is acceptable based on the test results and/or request that the subject undergo additional testing.

Thus, the six-minute walk test system enables the health care provider to analyze the test results at their convenience while also enabling the subject to perform the six-minute walk test at their convenience. Accordingly, the six-minute walk test system disclosed herein enables the health of the subject to be evaluated while reducing the burden of dealing with scheduling conflicts, removing the need for clinicians to sacrifice their time to administer the test, and removing expenses and/or difficulties that would otherwise be encountered as a result of going to a clinical setting for the test. Therefore, the six-minute walk test system disclosed herein improves the usability of the six-minute walk test to help evaluate the health of a subject.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly that might, for example, otherwise share a same name.

As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

As used herein, “programmable circuitry” is defined to include (i) one or more special purpose electrical circuits (e.g., an application specific circuit (ASIC)) structured to perform specific operation(s) and including one or more semiconductor-based logic devices (e.g., electrical hardware implemented by one or more transistors), and/or (ii) one or more general purpose semiconductor-based electrical circuits programmable with instructions to perform specific functions(s) and/or operation(s) and including one or more semiconductor-based logic devices (e.g., electrical hardware implemented by one or more transistors). Examples of programmable circuitry include programmable microprocessors such as Central Processor Units (CPUs) that may execute first instructions to perform one or more operations and/or functions, Field Programmable Gate Arrays (FPGAs) that may be programmed with second instructions to cause configuration and/or structuring of the FPGAs to instantiate one or more operations and/or functions corresponding to the first instructions, Graphics Processor Units (GPUs) that may execute first instructions to perform one or more operations and/or functions, Digital Signal Processors (DSPs) that may execute first instructions to perform one or more operations and/or functions, XPUs, Network Processing Units (NPUs) one or more microcontrollers that may execute first instructions to perform one or more operations and/or functions and/or integrated circuits such as Application Specific Integrated Circuits (ASICs). For example, an XPU may be implemented by a heterogeneous computing system including multiple types of programmable circuitry (e.g., one or more FPGAs, one or more CPUs, one or more GPUs, one or more NPUs, one or more DSPs, etc., and/or any combination(s) thereof), and orchestration technology (e.g., application programming interface(s) (API(s)) that may assign computing task(s) to whichever one(s) of the multiple types of programmable circuitry is/are suited and available to perform the computing task(s).

As used herein integrated circuit/circuitry is defined as one or more semiconductor packages containing one or more circuit elements such as transistors, capacitors, inductors, resistors, current paths, diodes, etc. For example an integrated circuit may be implemented as one or more of an ASIC, an FPGA, a chip, a microchip, programmable circuitry, a semiconductor substrate coupling multiple circuit elements, a system on chip (SoC), etc.

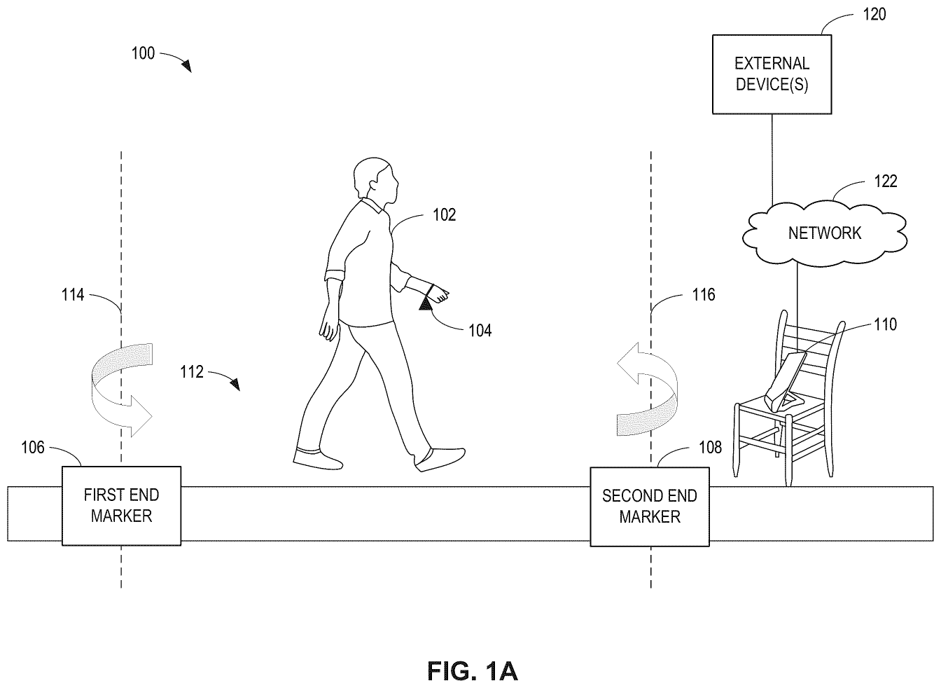

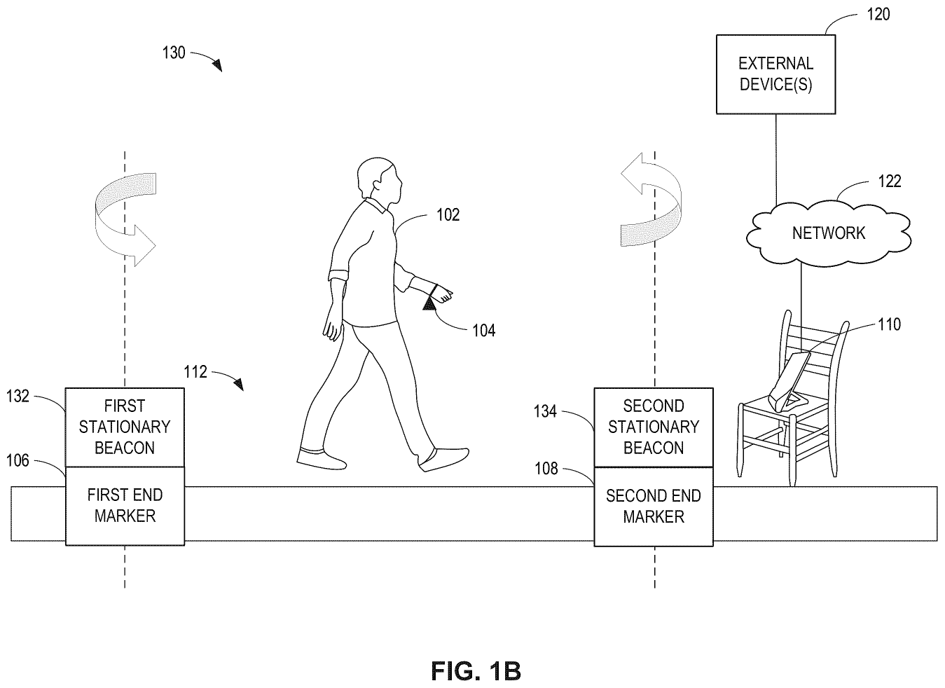

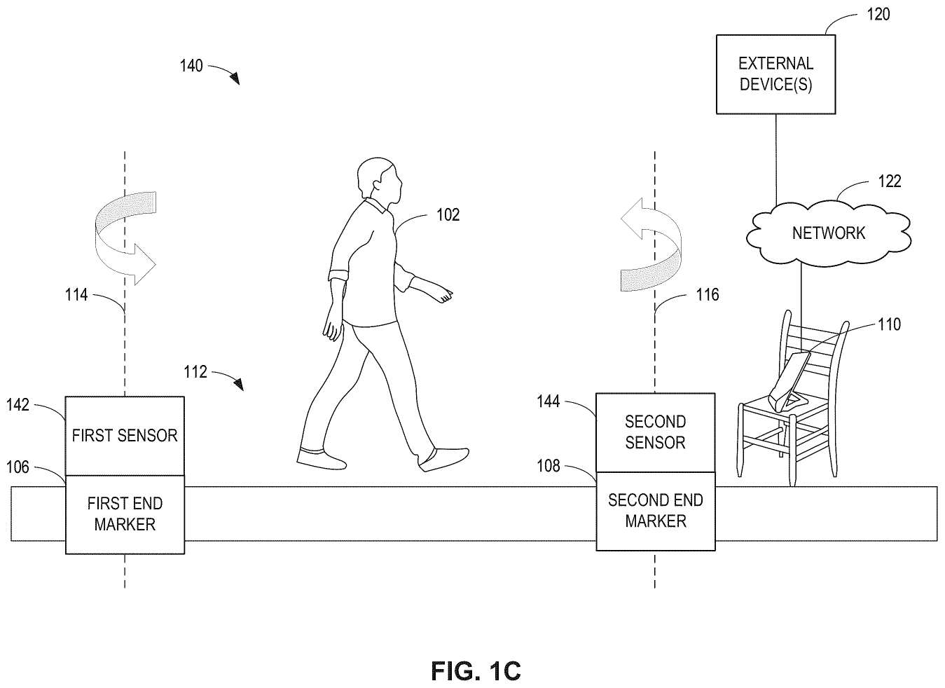

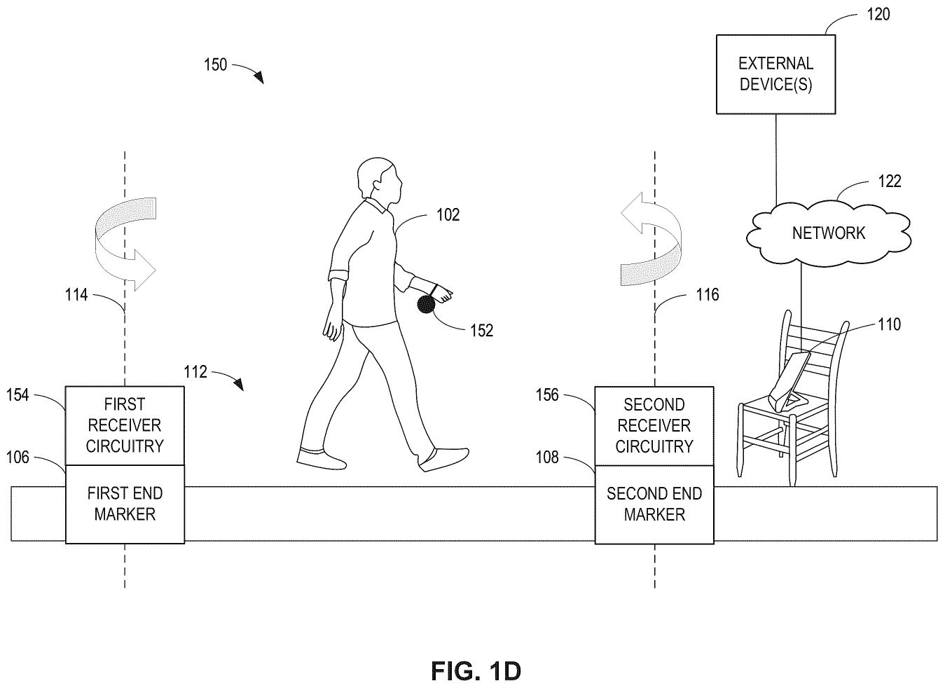

Turning to the figures, FIG. 1A is a first example six-minute walk test system 100 in accordance with teachings disclosed herein. In the illustrated example of FIG. 1A, the six-minute walk test system 100 includes a subject 102, a mobile beacon 104 carried by the subject 102, a first end marker 106, a second end marker 108, and a mobile device 110. In some examples, the first end marker 106 and the second end marker 108 are stationary objects, parts, or markings. For example, the first end marker 106 and the second end marker 108 can be cones, household items, ends of a string or rope, a marking on the floor, wall, or ceiling, etc. In some examples, the first end marker 106 and the second end marker 108 include stationary beacons. In some examples, the mobile beacon 104, the first end marker 106, and/or the second end maker 108 utilize Bluetooth communication. In other examples, other communication protocol and/or data exchange technologies may be used.

To set up the six-minute walk test, the subject can utilize the mobile device 110 (e.g., access an application on the mobile device 110), which provides instructions. For example, the mobile device 110 can instruct the subject to place the first end marker 106 and walk a certain distance away from the first end marker 106 (e.g., 20-40 feet) in a straight line before placing the second end marker 108 to define a track 112 (e.g., a course, a path, etc.) for the test. In some examples, the mobile device 110 instructs the subject to place the first end marker 106 and fully extend a material (e.g., a rope or string) connecting the end markers 106, 108 before placing the second end marker 108. In such examples, the length of the material is predetermined such that the length of the track 112 is known without the subject 102 having to measure the distance.

Further, the mobile device 110 can instruct the subject 102 to rest for a certain period before obtaining pre-test blood pressure, heart rate, and oxygen saturation measurements, which the subject 102 can record on the mobile device 110. In response to completing the pre-test measurements, the mobile device 110 instructs the subject 102 to wear the mobile beacon 104, start a countdown timer, place the mobile device 110 within a certain range of the track 112, and walk to either the first end marker 106 or the second end marker 108 to wait for the test to begin. In the illustrated example, the mobile device 110 instructs the subject 102 to place the mobile device 110 at an end of the track 112 outside of an area between a first plane 114 defined by the first end marker 106 and a second plane 116 defined by the second end marker 108. In the illustrated example of FIG. 1A, the mobile device 110 is placed near the second end marker 108 outside of the track 112. As such, the mobile device 110 receives a local minimum signal strength (e.g., attenuation) when the subject is at the first end marker 106 and receives a local maximum signal strength (e.g., attenuation) when the subject is at the second end marker 108. As used in these examples, attenuation refers to a signal strength.

In some examples, the mobile device 110 instructs the subject 102 to activate (i.e., turn on) the mobile beacon 104 to cause the mobile beacon 104 to emit signals. When the countdown timer expires, the mobile device 110 renders an audible and/or visual indication for the subject 102 to begin walking.

In some examples, when the end markers 106, 108 include stationary beacons, at a beginning of the six-minute walk test, the mobile device 110 identifies an approximate signal strength range associated with the track 112 based on a first signal strength received from the first end marker 106 (e.g., the first stationary beacon) and a second signal strength received from the second end marker 108 (e.g., the second stationary beacon). Specifically, the first signal strength from the first end marker 106 defines an approximate minimum signal strength associated with a first end of the track 112 and the second signal strength from the second end marker 108 defines an approximate maximum signal strength associated with a second end of the track 112. In such examples, the mobile device 110 ignores or discards the signals transmitted by the first end marker 106 and the second end marker 108 after identifying the signal strength range to enable more computing resources to be dedicated to other actions, such as tracking the mobile beacon 104.

In some examples, when the end markers 106, 108 do not include communication capabilities (e.g., when the end markers 106, 108 correspond to cones, household items, markings, etc.), the end markers 106, 108 do not emit signals and, thus, are not utilized to the approximate minimum signal strength and the approximate maximum signal strength to be expected during the test. Instead, the approximate minimum and maximum signal strengths may be determined based on the signal strengths received from the mobile beacon 104 during the test, as discussed in further detail below.

During the six-minute walk test, as the subject 102 travels back and forth between the first end marker 106 and the second end marker 108, the mobile device 110 measures a strength of the signals emitted by the mobile beacon 104. Specifically, the mobile device 110 identifies the signals transmitted by the mobile beacon 104 based on an identifier carried by the signals.

In some examples, the mobile beacon 104 transmits signals at a first frequency (e.g., once per second) and the mobile device 110 queries (e.g., transmits a request) for a signal from the mobile beacon 104 at a second frequency (e.g., twice per second). As a result, the mobile device 110 increases a frequency at which the mobile beacon 104 transmits signals and, in turn, improves an accuracy with which a relative position of the mobile beacon 104 can be determined. In some examples, the mobile device 110 periodically records the measured strength with a timestamp relative to the six-minute period for the test. When the mobile device 110 determines that the signal associated with the mobile beacon 104 is not being received or is being received with approximately the same signal strength for a certain period (e.g., 5 seconds), the mobile device 110 can prompt the subject 102 to ensure that they are carrying the mobile beacon 104, that the mobile beacon 104 has battery power, and/or that the mobile beacon 104 is powered on.

In response to an expiration of the six-minute period, the mobile device 110 transmits the raw signal strength data to one or more remote or external device(s) 120 via a network 122. For example, the external device(s) 120 can be a server or any other processor or programmable circuitry associated with a distributor of the six-minute walk test system 100. In FIG. 1A, the network 122 is implemented as a public network such as for example, the Internet. However, any other type of networks (e.g., wired/cabled, wireless, mobile cellular, etc.) which may be public or private, and any combination thereof may additionally and/or alternatively be used. Additionally, although the example six-minute walk test system 100 utilizes the network 122 for communications between the mobile device 110 and the other external device(s) 120, it should be understood that the mobile device 110 and the other device(s) 120 can communicate using any alternative forms of communication. In some examples, the mobile device 110 stores the test results in a memory of the mobile device 110. In such examples, the subject 102 brings the mobile device 110 to a subsequent meeting with their health care provider where the health care provider can access and/or download the test results stored in the memory of the mobile device 110 to, for example, the external device 120.

In the illustrated example of FIG. 1A, the external device(s) 120 process the raw signal strength data to determine the distance that the subject 102 traveled during the six-minute walk test. The external device(s) 120 normalize the raw strength data and remove any outliers. For example, the external device(s) 120 remove noise from the signal strength data, such as by computing a moving average (e.g., a 3-point moving average, a 5-point moving average, a 7-point moving average, etc.) for each signal strength data point.

In the illustrated example, the external device(s) 120 identify a quantity of traversals (e.g., trips) that the subject completed between the end markers 106, 108 based on changes in the slope of the signal strength data. In some examples, the external device(s) 120 determine a traversal was completed (e.g., the subject 102 traveled from the first end marker 106 to the second end marker 108, the subject traveled from the second end marker 108 to the first end marker 106) in response to encountering a change between increasing and decreasing signal strength (e.g., a change between positive and negative signal strength over time slope). In such examples, the external device(s) 120 verify that the change is indicative of the subject 102 turning around at an end of the track 112 in response to a signal strength after the change being different from a local minimum or maximum encountered before the change by at least a strength threshold (e.g., 5 dB). Specifically, as the subject 102 travels from the first end marker 106 to the second end marker 108, the signal strength that the mobile device 110 receives from the mobile beacon 104 increases. Further, when the subject 102 reaches the second end marker 108 and begins to travel back to the first end marker 106, the signal strength over time stops increasing and begins decreasing and, thus, the slope changes from positive to negative because the subject 102 reached a location in the track 112 closest to the mobile device 110 and then began moving away from the mobile device 110. Similarly, when the subject 102 reaches the first end marker 106 and begins to travel back to the second end marker 108, the slope of the signal strength over time changes from negative to positive as the subject 102 begins to move towards the mobile device 110. By utilizing a strength change threshold to identify instances when the subject 102 turned around at one of the end markers 106, 108, the external device(s) 120 improve an accuracy of a quantity traversals counted. For example, the external device(s) 120 prevent noise, natural variance, and/or changes in slope caused by the subject 102 swinging an arm that carries the mobile beacon 104 from being flagged as the subject 102 reaching one of the end markers 106, 108.

In some examples, in response to an increase in the signal strength satisfying (e.g., being greater than, being greater than or equal to) a threshold increase (e.g., a 5 dB increase) when the signal strength had been decreasing for a certain number of datapoints prior to satisfying the threshold increase, the external device(s) 120 identify a local minimum signal strength encountered prior to the threshold increase being satisfied. The external device(s) 120 associate the local minimum with the subject 102 being at the first end marker 106. In some examples, in response to a decrease in the signal strength satisfying (e.g., being less than, being less than or equal to) a threshold decrease (e.g., a 5 dB decrease) when the signal strength had been increasing for a certain number of datapoints prior to satisfying the threshold decrease, the external device(s) 120 identify a local maximum signal strength encountered prior to the threshold decrease. Further, the external device(s) 120 associate the local maximum with the subject 102 being at the second end marker 108. In the illustrated example of FIG. 1A, the external device(s) 120 count the local maximums and minimums encountered or the threshold increases and decreases satisfied to determine a quantity of traversals that the subject 102 completed between the first end marker 106 and the second end marker 108 during the six-minute period.

In some examples, the external device(s) 120 adjusts the threshold increase and/or the threshold decrease that corresponds to the subject changing direction based on an estimated speed of the subject 102. For example, the external device(s) 120 can estimate the speed of the subject based on a slope corresponding to the change in the signal strength that the mobile device 110 received from the mobile beacon 104 over time. As discussed above, the strength of the signal emitted by Bluetooth beacons inherently varies. That is, even when the mobile beacon 104 is not moving, the mobile device 110 may receive different signal strengths from the mobile beacon 104 at different times. This variance can be ignored or mitigated when the subject 102 is moving at a speed that satisfies (e.g., is greater than, is greater than or equal to) a speed threshold as a potential signal strength range that results from the natural variance in the signal strength emitted by the mobile beacon 104 changes with the movement of the mobile beacon 104.

For example, the natural variance of the strength of the signals emitted by the mobile beacon 104 can be plus or minus (±) 8 dB. Additionally, the strength of the signal emitted by the mobile beacon 104 at a first location can be 40±8 dB; the strength of the signal emitted by the mobile beacon 104 at a second location can be 50±8 dB; and the strength of the signal emitted by the mobile beacon 104 at a third location farther than the second location from the first location can be 55±8 dB. Accordingly, in this example, the third location is closer than the first location and the second location to the mobile device 110. In this example, the subject 102 is traveling from the first location towards the mobile device 110. When the subject 102 travels from the first location to the third location between two signal emissions, the maximum reduction in signal strength that could be encountered as a result of the natural variance in the signal strength emitted by the mobile beacon 104 is 1 dB. Further, as a reduction of 1 dB over a certain period of time does not satisfy the example 5 dB threshold decrease that the external device(s) 120 associates with the subject turning around, this movement would not be flagged as the subject turning around. Alternatively, when the subject 102 travels from the first location to the second location between the certain number of signal emissions, the maximum reduction in signal strength that could be encountered as a result of the natural variance in the signal strength emitted by the mobile beacon 104 is 6 dB. As a reduction of 6 dB over a certain period of time satisfies the example 5 dB threshold decrease that the external device(s) 120 associates with the subject 102 turning around, the external device(s) 120 would mistake the 6 dB as meaning that the subject 102 turned around and started traveling away from the mobile device 110. That is, the external device(s) 120 would generate a false positive turnaround determination. Thus, movement of the mobile beacon 104 between two signal emissions that satisfies (e.g., is greater than, is greater than or equal to) a distance threshold will prevent variance beyond the threshold increase or the threshold decrease from being encountered. However, movement of the mobile beacon 104 between two or more signal emissions that does not satisfy (e.g., is less than or equal to, is less than) the distance threshold necessitates an increase in the threshold increase and the threshold decrease associated with the subject turning around. Accordingly, the external device(s) 120 estimates a speed of the subject 102 based on a slope of the signal strength received by the mobile device 110 over time. In FIG. 1A, one or more slope thresholds are associated with different turnaround thresholds (e.g., threshold increase and the threshold decrease). As such, the external device(s) 120 can compare the absolute value of the slope to the slope threshold(s) to determine which turnaround threshold to utilize.

In some examples, when the six-minute period for the test expires after the subject has started but not completed a traversal, the external device(s) 120 determine a distance that the subject traveled during the partially completed traversal based on a ratio of the signal strength change encountered since the last local maximum or minimum and an average signal strength range encountered during complete traversals between the end markers 106, 108. In some examples, the external device identifies an incomplete trip between the end markers 106, 108 in response to an identified local minimum or maximum being outside of a threshold range of an average local minimum or maximum for the test. For example, the external device(s) 120 can determine the subject turned around before reaching the first end marker 106 in response to an identified local minimum being greater than the average local minimum by at least a predetermined threshold (e.g., 15 dB). Further, the external device(s) 120 can determine the distance associated with any incomplete trips based on a ratio between the average signal strength range encountered during complete traversals and a change in the signal strength encountered during the partially completed traversal. Further, the external device(s) 120 can multiply the predetermined distance of the track 112 by the number of complete and partially completed traversals to calculate the total distance traveled by the subject during the six-minute walk test. In the illustrated example, the external device(s) 120 transmit the total distance traveled during the six-minute walk test to the mobile device 110.

In the illustrated example of FIG. 1A, after the six-minute period, the mobile device 110 can instruct the subject 102 to measure and record post-test vital signs. Further, the mobile device 110 can present the total distance traveled to the subject 102 via a display. In some examples, the mobile device 110 presents the total distance traveled and/or the measured vital signs in comparison to previous test results, so the subject 102 can identify any relative progress achieved. Additionally, the mobile device 110 can prompt the subject 102 to indicate how they felt during and/or after the test. In some examples, the mobile device 110 transmits the test results (e.g., the total distance traveled, the measured vital signs, responses from the subject 102) to one or more other device(s) 120 via a network 122.

FIG. 1B is another example six-minute walk test system 130 in accordance with teachings disclosed herein. The example six-minute walk test system 130 of FIG. 1B includes the mobile beacon 104 carried by the subject 102, the first end marker 106, the second end marker 108, the mobile device 110, the track 112, the external device(s) 120, and the network 122 of FIG. 1A. In the illustrated example of FIG. 1B, the six-minute walk test system 130 includes a first stationary beacon 132 and a second stationary beacon 134. More particularly, the first stationary beacon 132 is positioned on or adjacent to the first end marker 106, and the second stationary beacon 134 is positioned on or adjacent to the second end marker 108. In some examples, the first stationary beacon 132 and the second stationary beacon 134 serve as (e.g., replace) the first end marker 106 and the second end marker 108.

In the illustrated example of FIG. 1B, the first stationary beacon 132 and the second stationary beacon 134 transmit signals. The signals can carry an identifier indicative of the beacon 132, 134 from which the signal originated. In turn, the mobile device 110 receives the signal and a corresponding signal strength. In some examples, the mobile device 110 triggers (e.g., pings) the stationary beacons 132, 134 to transmit signals prior to the subject 102 starting the six-minute walk test. In some examples, the mobile device 110 identifies a signal strength range associated with the track 112 based on the signals from the stationary beacons 132, 134. As a result, the mobile device 110 can reject signals outside of the signal strength range. For example, the mobile device 110 can configure a bandpass filter based on the signal strength associated with the track 112. Further, the mobile device 110 can detect the signals emitted by the mobile beacon 104 based on the signal strength range associated with the track 112. Advantageously, the signal strength range identified by the mobile device 110 at the beginning of the test can minimize or otherwise reduce signals that the mobile device 110 processes in search of the signal from the mobile beacon 104. In some examples, the mobile device 110 indicates the signal strength range to the external device(s) 120. In some examples, the external device(s) 120 utilize the signal strength range to identify when the signal strength from the mobile beacon 104 corresponds to the subject 102 being at the corresponding end marker 106, 108.

FIG. 1C is another example six-minute walk test system 140 in accordance with teachings disclosed herein. The example six-minute walk test system 140 of FIG. 1C includes the subject 102, the first end marker 106, the second end marker 108, the mobile device 110, the track 112, the external device(s) 120, and the network 122 of FIGS. 1A-1B. In the illustrated example of FIG. 1C, the six-minute walk test system 140 includes a first sensor 142 and a second sensor 144. More particularly, the first sensor 142 is positioned on or adjacent to the first end marker 106, and the second sensor 144 is positioned on or adjacent to the second end marker 108. In some examples, the first sensor 142 and the second sensor 144 serve as (e.g., replace) the first end marker 106 and the second end marker 108.

Although the illustrated example of FIG. 1C includes a first sensor 142 and a second sensor 144, it should be understood that a different quantity of sensors can be utilized by the six-minute walk test system 140. For example, the six-minute walk test system 140 can include the first sensor 142 or the second sensor 144 and not both. Alternatively, the six-minute walk test system 140 can include a plurality of the sensors 142, 144 spaced apart along a distance of the track 112.

In some examples, the first sensor 142 and the second sensor 144 are proximity or sonar sensors. In such examples, as the subject 102 approaches the first sensor 142 or the second sensor 144, an ultrasonic sound wave emitted by the sensor 142, 144 reflects off the patient and returns to the sensor 142, 144. In such examples, the mobile device 110 provides time stamp data associated with the transmission and reception of signals by the sensors 142, 144 to the external device(s) 120, which identifies locations of the subject 102 during the six-minute walk test based on a difference between the time that the signal was emitted and the time that the signal was received by the sensors 142, 144.

In some examples, the first sensor 142 and the second sensor 144 are infrared sensors. In such examples, as the subject 102 traverses the track 112, the sensors 142, 144 monitor infrared radiation in the electromagnetic spectrum (e.g., light reflected by and heat emitted by the subject). When the infrared radiation satisfies (e.g., is greater than, is greater than or equal to) a threshold, the sensors 142, 144 can generate an indication (e.g., transmit a signal to the mobile device 110) corresponding to the subject reaching the location associated with the sensor 142, 144 (e.g., the end of the track 112). In some examples, the signal transmitted by the sensors 142, 144 includes an identifier that the mobile device 110 and/or the external device(s) 120 associates with a location of the sensor 142, 144. In some examples, the signal includes a timestamp and/or the mobile device 110 generates a timestamp in response to receiving the signal. Accordingly, the external device(s) 120 can analyze the signal data to identify the location of the subject 102 at various instances throughout the six-minute walk test and, in turn, a distance that the subject 102 traveled during the six-minute walk test.

In some examples, the first sensor 142 and the second sensor 144 are optical sensors that capture images of the subject 102 during the six-minute walk test. In such examples, the mobile device 110 and/or the external device(s) 120 can process the images to determine the distance between the subject 102 and the end marker 106, 108 associated with the sensor 142, 144 and, in turn, a distance that the subject 102 traveled along the track 112 during the test.

FIG. 1D is another example six-minute walk test system 150 in accordance with teachings disclosed herein. The example six-minute walk test system 150 of FIG. 1D includes the subject 102, the first end marker 106, the second end marker 108, the mobile device 110, the track 112, the external device(s) 120, and the network 122 of FIGS. 1A-1C. In the illustrated example of FIG. 1D, the six-minute walk test system 150 includes transmitter circuitry 152 (e.g., a Wi-Fi or radio transmitter) carried by the subject 102, first receiver circuitry 154, and second receiver circuitry 156. More particularly, the first receiver circuitry 154 is positioned on or adjacent to the first end marker 106, and the second receiver circuitry 156 is positioned on or adjacent to the second end marker 108. In some examples, the first receiver circuitry 154 and the second receiver circuitry 156 serve as (e.g., replace) the first end marker 106 and the second end marker 108.

In the illustrated example of FIG. 1D, the transmitter circuitry 152 transmits signals (e.g., Wi-Fi or radio signals) as the subject 102 traverses the track 112. The first and second receiver circuitry 154, 156 receive the signals transmitted by the transmitter circuitry 152. In some examples, the first and second receiver circuitry 154, 156 store a strength of the signal and/or transmit a signal indicative of the strength of the signal to the mobile device 110. In such examples, the mobile device 110 and/or the external device(s) 120 can determine the location of the subject 102 throughout the test based on the strengths of the signals and, in turn, determine the distance that the subject 102 traveled during the test.

In some examples, the transmitter circuitry 152 stores timestamp data indicative of when the transmitter circuitry 152 transmitted signals. In some examples, the signals transmitted by the transmitter circuitry 152 carries timestamp information. The mobile device 110, the external device(s) 120, and/or the first and second receiver circuitry 154, 156 can associate the time of signal transmission with the time of signal reception. The external device(s) 120 can determine the location of the subject 102 throughout the test based on the time that the transmitted signals took to reach the receiver circuitry 154, 156. As a result, the external device(s) 120 can determine the distance that the subject 102 traveled during the test.

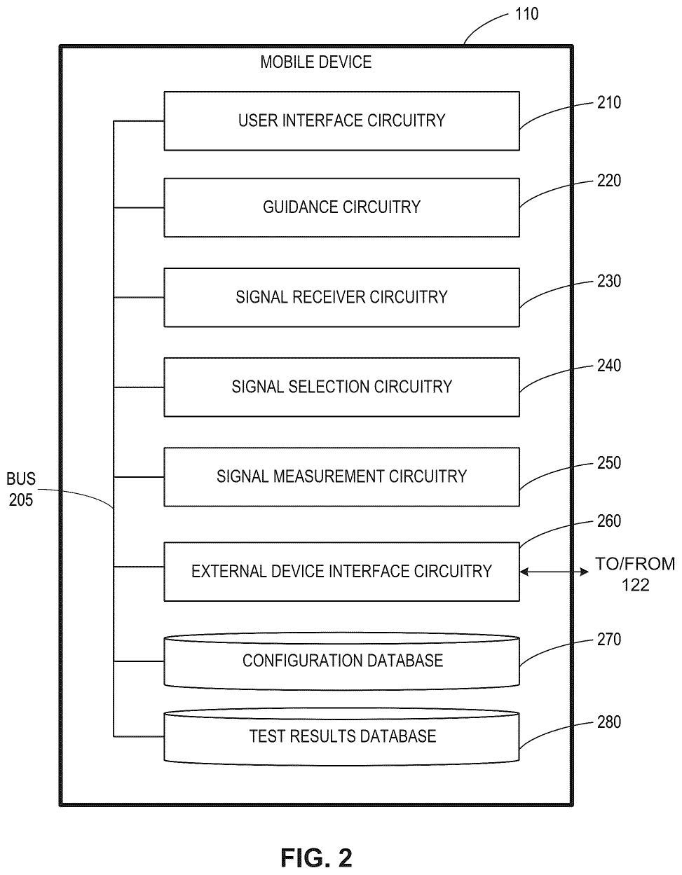

FIG. 2 is a block diagram of an example implementation of the mobile device 110 to measure raw signal strength data during the six-minute walk test. The mobile device 110 of FIG. 2 may be instantiated (e.g., creating an instance of, bring into being for any length of time, materialize, implement, etc.) by programmable circuitry such as a central processing unit executing instructions. Additionally or alternatively, the mobile device 110 of FIG. 2 may be instantiated (e.g., creating an instance of, bring into being for any length of time, materialize, implement, etc.) by an ASIC or an FPGA structured to perform operations corresponding to the instructions. It should be understood that some or all of the circuitry of FIG. 2 may, thus, be instantiated at the same or different times. Some or all of the circuitry may be instantiated, for example, in one or more threads executing concurrently on hardware and/or in series on hardware. Moreover, in some examples, some or all of the circuitry of FIG. 2 may be implemented by microprogrammable circuitry executing instructions to implement one or more virtual machines and/or containers.

The example mobile device 110 of FIG. 2 includes an example bus 205, example user interface circuitry 210, example guidance circuitry 220, example signal receiver circuitry 230, example signal selection circuitry 240, example signal measurement circuitry 250, example external device interface circuitry 260, an example configuration database 270, and an example test results database 280. Examples disclosed herein reference Bluetooth low energy signals, which also is known as Bluetooth LE or BLE. However, other examples may use other communication and/or data transfer technologies.

In the illustrated example of FIG. 2, the user interface circuitry 210, the guidance circuitry 220, the signal receiver circuitry 230, the signal selection circuitry 240, the signal measurement circuitry 250, the external device interface circuitry 260, the configuration database 270, and the test results database 280 are in communication with the bus 205. In some examples, the bus 205 can be implemented with bus circuitry, bus software, and/or bus firmware. For example, the bus 205 can be implemented by at least one of an Inter-Integrated Circuit (I2C) bus, a Serial Peripheral Interface (SPI) bus, a Peripheral Component Interconnect (PCI) bus, or a Peripheral Component Interconnect Express (PCIe or PCIE) bus. Additionally or alternatively, the bus 205 can be implemented by any other type of computing or electrical bus.

The mobile device 110 of FIG. 2 includes the user interface circuitry 210 to enable the subject 102 to access instructions, access test results, provide responses to prompts, and/or indicate pre- and post-test vital signs. For example, the user interface circuitry 210 can be implemented by a display and/or a speaker to deliver instructions and indicate test results to the subject 102. Additionally or alternatively, the user interface circuitry 210 can be implemented by a touchscreen, a keyboard, a microphone, and/or any other devices that enable the subject 102 to input vital measurements and/or responses to prompts.

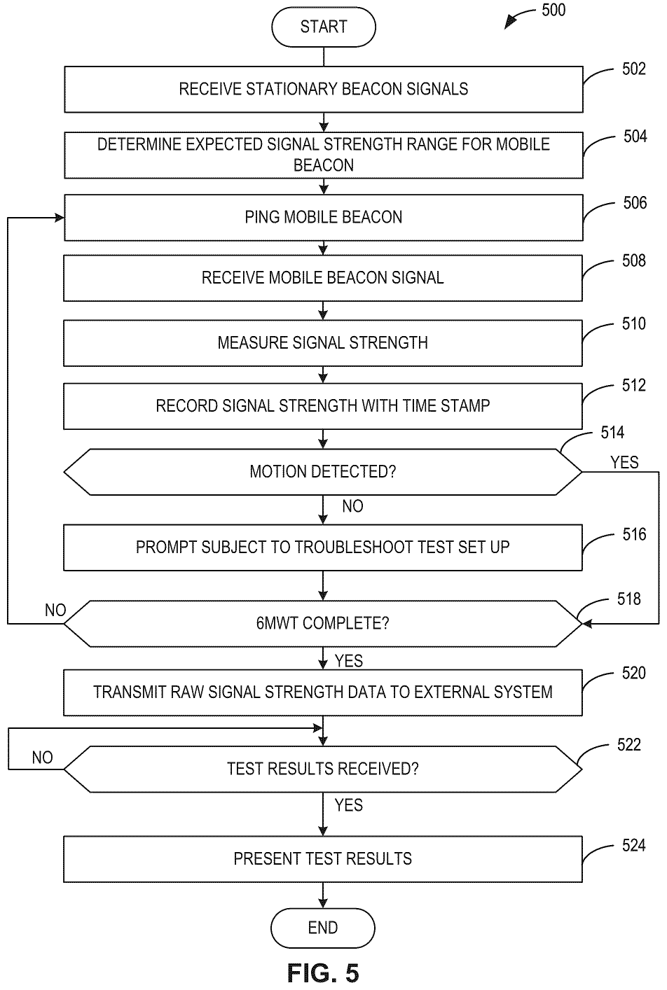

The mobile device 110 of FIG. 2 includes the guidance circuitry 220 to generate instructions to help the subject 102 perform the six-minute walk test. For example, the guidance circuitry 220 can generate instructions on how to prepare the equipment, measure the walking track, rest before the test, perform the test, measure vital signs before and after the test, and/or provide relevant information before, during, and/or after the test. Further, the guidance circuitry 220 can cause the generated instructions to be audibly and/or visually presented via the user interface circuitry 210. In some examples, the guidance circuitry 220 is instantiated by programmable circuitry executing subject guiding instructions and/or configured to perform operations such as those represented by the flowchart of FIG. 5.

The mobile device 110 of FIG. 2 includes the signal receiver circuitry 230 to receive signals (e.g., Bluetooth signals, radio signals, Wi-Fi signals, etc.) transmitted by the mobile beacon 104, the first stationary beacon 132, and the second stationary beacon 134, the first sensor 142, the second sensor 144, the transmitter circuitry 152, the first receiver circuitry 154, and/or the second receiver circuitry 156. The signals can include an identifier indicative of the respective beacon 104, 132, 134, sensor 142, 144, or other circuitry 152, 154, 156 where the signal originated. In some examples, the guidance circuitry 220 instructs the signal receiver circuitry 230 to receive the signals in response to the six-minute walk test being in progress.

The mobile device 110 of FIG. 2 includes the signal selection circuitry 240 to identify the received signal (e.g., Bluetooth signal) that was transmitted by the mobile beacon 104, the first stationary beacon 132, and the second stationary beacon 134, the first sensor 142, the second sensor 144, the transmitter circuitry 152, the first receiver circuitry 154, and/or the second receiver circuitry 156. For example, the signal selection circuitry 240 can identify the signal that originated from the mobile beacon 104 based on the identifiers received with the respective signals. Specifically, the signal selection circuitry 240 can determine the identifier associated with the mobile beacon 104 based on data indicative of the identifier for the mobile beacon 104 stored in the configuration database 270. In turn, the signal selection circuitry 240 transmits a signal indicative of the signal associated with the mobile beacon 104 to the signal measurement circuitry 250. In some examples, the signal selection circuitry 240 determines an expected signal strength range associated with the mobile beacon 104 based on respective strengths of signals received from the respective stationary beacons 132, 134 at a start of the six-minute walk test. The signal selection circuitry 240 can discard or cause the signal receiver circuitry 230 to ignore the signals from the stationary beacons 132, 134 after the expected signal strength range has been determined. As such, the signal selection circuitry 240 minimizes or otherwise reduces a quantity of signals processed to identify the signal associated with the mobile beacon 104, which can save processing resources including bandwidth and battery power for the mobile device 110. In some examples, the signal selection circuitry 240 is instantiated by programmable circuitry executing signal selection instructions and/or configured to perform operations such as those represented by the flowchart of FIG. 5.

The mobile device 110 of FIG. 2 includes the signal measurement circuitry 250 to measure a strength (e.g., an attenuation) of the signal received from the mobile beacon 104. In some examples, the signal measurement circuitry 250 records the measured signal strength with a timestamp via the test results database 280. For example, the timestamp can be indicative of a time relative to the six-minute walk test period. In some examples, the signal measurement circuitry 250 records data associated with signals received from the first sensor 142, the second sensor, 144, the transmitter circuitry 152, the first receiver circuitry 154, and/or the second receiver circuitry 156 via the test results database 280. In some examples, the signal measurement circuitry 250 is instantiated by programmable circuitry executing signal measurement instructions and/or configured to perform operations such as those represented by the flowchart of FIG. 5.

The mobile device 110 of FIG. 2 includes the external device interface circuitry 260 to transmit data to and receive data from the external device(s) 120 of FIGS. 1A-1D. For example, the external device interface circuitry 260 transmits data to and receives data from the external device(s) 120 via the network 122 of FIGS. 1A-1D. In some examples, the external device interface circuitry 260 transmits the data stored via the test results database 280 to the external device(s) 120. For example, the external device interface circuitry 260 transmits raw signal strength data received by the signal measurement circuitry 250 to the external device(s) 120 of FIGS. 1A-1D. Additionally, the external device interface circuitry 260 receives a total distance that the subject 102 traveled during the six-minute walk test from the external device(s) 120. In some examples, the external device interface circuitry 260 receives a confidence score associated with the determined total distance traveled and/or prompts to be provided to the subject 102 based on the determined confidence score and/or total distance traveled from the external device(s) 120.

The mobile device 110 of FIG. 2 includes the configuration database 270 to store data indicative of the identifier carried by respective signals that the mobile beacon 104, the first stationary beacon 132, the second stationary beacon 134, the first sensor 142, the second sensor 144, the transmitter circuitry 152, the first receiver circuitry 154, and/or the second receiver circuitry 156 transmit. In some examples, the configuration database 270 stores data indicative of a predetermined distance associated with the track 112. For example, the configuration database 270 can include data that indicates a certain change in signal strength corresponds to movement of the subject 102 over a certain and/or corresponding distance. The mobile device 110 of FIG. 2 includes the test results database 280 to store data associated with the total distance that the subject 102 traveled during the six-minute walk test, pre-test vitals, post-test vitals, and/or responses to prompts provided to the subject 102 before and/or after the six-minute walk test.

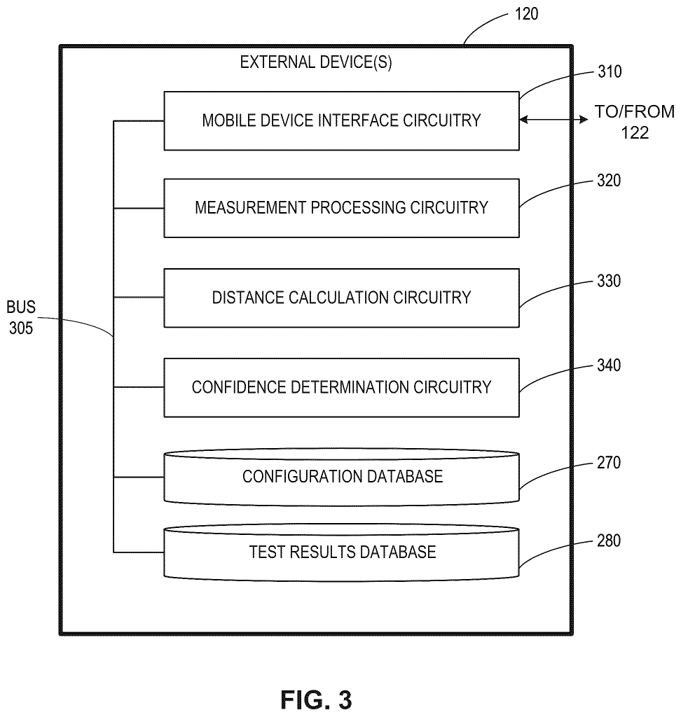

FIG. 3 is a block diagram of an example implementation of the external device(s) 120 to determine a distance that the subject 102 traveled during the six-minute walk test. The external device(s) 120 of FIG. 3 may be instantiated (e.g., creating an instance of, bring into being for any length of time, materialize, implement, etc.) by programmable circuitry such as a central processing unit executing instructions. Additionally or alternatively, the external device(s) 120 of FIG. 3 may be instantiated (e.g., creating an instance of, bring into being for any length of time, materialize, implement, etc.) by an ASIC or an FPGA structured to perform operations corresponding to the instructions. It should be understood that some or all of the circuitry of FIG. 3 may, thus, be instantiated at the same or different times. Some or all of the circuitry may be instantiated, for example, in one or more threads executing concurrently on hardware and/or in series on hardware. Moreover, in some examples, some or all of the circuitry of FIG. 3 may be implemented by microprogrammable circuitry executing instructions to implement one or more virtual machines and/or containers.

The external device(s) 120 of FIG. 3 include an example bus 305, example mobile device interface circuitry 310, example measurement processing circuitry 320, example distance calculation circuitry 330, and example confidence determination circuitry 340. In some examples, the external device(s) 120 of FIG. 3 also include the configuration database 270 and/or the test results database 280 of FIG. 2. In the illustrated example of FIG. 3, the mobile device interface circuitry 310, the measurement processing circuitry 320, the distance calculation circuitry 330, the confidence determination circuitry 340, the configuration database 270, and the test results database 280 are in communication with the bus 305. In some examples, the bus 305 can be implemented with bus circuitry, bus software, and/or bus firmware. For example, the bus 305 can be implemented by at least one of an Inter-Integrated Circuit (I2C) bus, a Serial Peripheral Interface (SPI) bus, a Peripheral Component Interconnect (PCI) bus, or a Peripheral Component Interconnect Express (PCIe or PCIE) bus. Additionally or alternatively, the bus 305 can be implemented by any other type of computing or electrical bus.

The external device(s) 120 of FIG. 3 include the mobile device interface circuitry 310 to transmit data to and receive data from the mobile device 110 of FIGS. 1A-1D and 2. For example, the mobile device interface circuitry 310 transmits data to and receives data from the mobile device 110 via the network 122 of FIGS. 1A-1D. The mobile device interface circuitry 310 receives data (e.g., raw signal strength data from the mobile beacon 104, etc.,) that the mobile device 110 received from the mobile beacon 104, the first stationary beacon 132, the second stationary beacon 134, the first sensor 142, the second sensor 144, the transmitter circuitry 152, the first receiver circuitry 154, and/or the second receiver circuitry 156 during the six-minute walk test. The mobile device interface circuitry 310 transmits the total distance that the subject 102 traveled during the six-minute walk test to the mobile device 110. In some examples, the mobile device interface circuitry 310 transmits a confidence score attributed to the determined total travel distance. In some examples, the mobile device interface circuitry 310 transmits instructions to the mobile device 110 in an attempt to improve the confidence score associated with subsequent test results. For example, the instructions can suggest that the subject 102 utilize a different testing environment and/or replace one or more of the beacons 104, 132, 134.

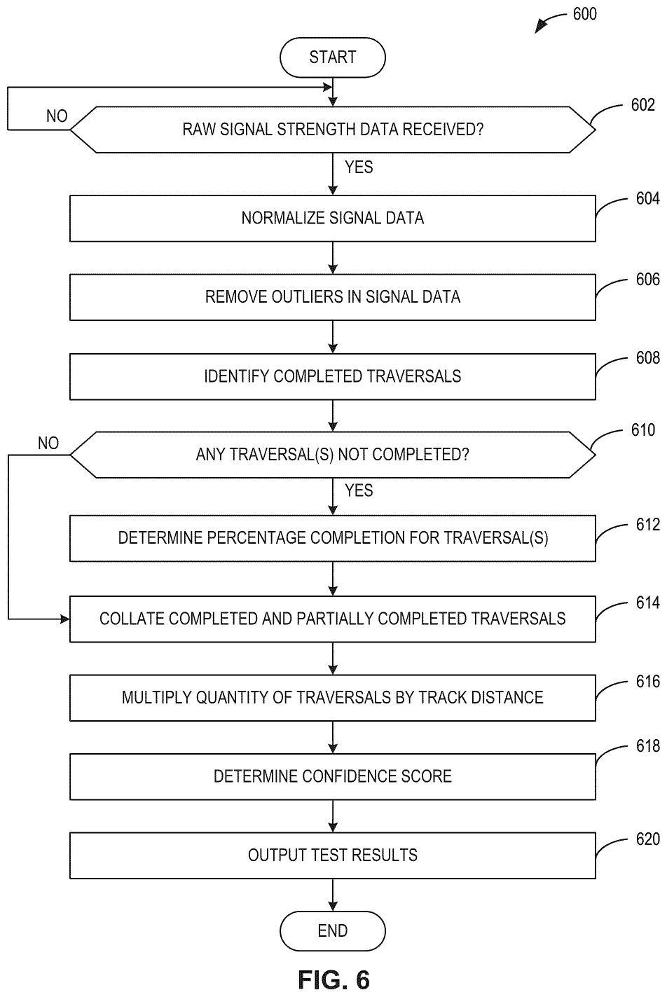

The external device(s) 120 of FIG. 3 include the measurement processing circuitry 320 to process the data that the mobile device 110 measured during the six-minute walk test. For example, the measurement processing circuitry 320 can normalize the signal strength data. Additionally, the measurement processing circuitry 320 can remove any outliers in the signal strength data. In some examples, the measurement processing circuitry 320 performs data smoothing by computing a moving average (e.g., a 3-point moving average, a 5-point moving average, a 7-point moving average, etc.) for each signal strength data point to remove the outliers. In some examples, the measurement processing circuitry 320 processes image data to determine a location of the subject 102 associated with the image data. In some examples, the measurement processing circuitry 320 is instantiated by programmable circuitry executing measurement processing instructions and/or configured to perform operations such as those represented by the flowchart of FIG. 6.

The external device(s) 120 of FIG. 3 includes the distance calculation circuitry 330 to determine a total distance that the subject 102 traveled during the six-minute walk test. For example, the distance calculation circuitry 330 can determine a total distance that the subject 102 traveled during the six-minute walk test based on the processed signal strength measurements. In some examples, the distance calculation circuitry 330 identifies a quantity of trips that the subject 102 completed between the first end marker 106 and the second end marker 108 based on a pattern in the processed signal strength measurements. For example, the distance calculation circuitry 330 can identify a quantity of traversals (e.g., trips, etc.) that the subject 102 completed between the end markers 106, 108 based on changes in the slope of the signal strength data.

In some examples, the distance calculation circuitry 330 identifies changes between positive and negative slope in the signal strength over time. After identifying a change between positive and negative slope in the signal strength data, the distance calculation circuitry 330 compares a local maximum or minimum at the change to one or more signal strength data points associated with a time following the change to determine whether the change in slope was a result of the subject 102 turning around at one of the end markers 106, 108 or a result of noise or random variance in the signal strength received by the mobile device 110. For example, the distance calculation circuitry 330 can determine whether a signal strength change in a predetermined number of data points following the change in slope satisfies (e.g., is greater than, is greater than or equal to) a difference threshold (e.g., 5 dB). In some examples, the distance calculation circuitry 330 determines the difference threshold based on a slope of the signal strength data. In some examples, when a quantity periodic rests that the subject 102 took during the test satisfies (e.g., is greater than, is greater than or equal to) a threshold, the distance calculation circuitry 330 infers that the subject is a relatively slow walker and adjusts (e.g., increases) the threshold change in signal strength associated with a turnaround.

In some examples, when the signal strength change satisfies the difference threshold, the distance calculation circuitry 330 compares the local maximum or minimum signal strength identified at the signal strength change to a signal strength range associated with the track and/or an average local maximum or minimum signal strength measured during the test. In response to the identified local maximum or minimum being within a threshold range (e.g., 15 dB) of the respective maximum or minimum defined by signal strength range associated with the track and/or the average local maximum or minimum signal strength measured during the test, the distance calculation circuitry 330 determines that the identified local maximum or minimum is indicative of the subject 102 being at one of the end markers 106, 108.

In some examples, when the identified local maximum or minimum is a first identified local maximum or minimum in the six-minute test period, the distance calculation circuitry 330 determines whether an initial signal strength measured before the first identified local maximum or minimum is within the threshold range of the respective maximum or minimum defined by signal strength range associated with the track and/or the average local maximum or minimum signal strength measured during the test. For example, when the first identified local maximum or minimum is a local minimum, the distance calculation circuitry 330 can determine whether the initial signal strength measurement is within the threshold range of the maximum defined by the signal strength range associated with the track and/or the average local maximum to determine whether the subject 102 started at the second stationary beacon 134 or in a middle portion of the track 112 (e.g., at a location between the first plane 114 and the second plane 116). Specifically, when the initial signal strength measurement is within the threshold range of the maximum defined by the signal strength range associated with the track and/or the average local maximum, the distance calculation circuitry 330 determines that the subject 102 completed a full traversal between the end markers 106, 108. In some examples, when the initial signal strength measurement is not within the threshold range of the maximum defined by the signal strength range associated with the track and/or the average local maximum, the distance calculation circuitry 330 determines that the subject 102 began the test in the middle portion of the track 112. In such examples, the distance calculation circuitry 330 determines a fraction or percentage of the track 112 that subject 102 traveled based on a ratio of (a) a signal strength difference between the initial signal strength measurement and the identified local maximum and (b) an average signal strength difference for a complete traversal. Similarly, when the identified local maximum or minimum is a last identified local maximum or minimum, the distance calculation circuitry 330 determines a fraction or percentage of the track 112 that the subject 102 traveled during the last traversal based on a ratio of (a) the signal strength difference between the last signal strength measurement and the last local maximum or minimum and (b) the average signal strength difference for a complete traversal.

In some examples, in response to the identified local maximum or minimum not being within the threshold range (e.g., 15 dB) of the respective maximum or minimum defined by signal strength range associated with the track and/or the average local maximum or minimum signal strength measured during the test, the distance calculation circuitry 330 can determine the subject 102 did not complete the traversal (e.g., turned around short of the respective end marker 106, 108 to which they were heading). In such examples, the distance calculation circuitry 330 determines a fraction or percentage of the track 112 that the subject 102 traveled during the traversals that were partially completed based on a ratio between (a) the signal strength difference between the local maximum and minimum associated with the partially completed traversal and (b) the average signal strength difference for a complete traversal.

In the illustrated example of FIG. 3, to calculate the total distance that the subject traveled during the six-minute walk test, the distance calculation circuitry 330 adds a quantity of traversals that the subject 102 completed—including fractions or percentages for partially completed traversals—and multiplies the quantity of traversals by the predetermined distance associated with the track 112. The distance calculation circuitry 330 causes the mobile device interface circuitry 310 to transmit the total distance that the subject 102 traveled to the mobile device 110. In some examples, the distance calculation circuitry 330 stores the total distance that the subject 102 traveled and/or the processed signal strength data via the test results database 280 with an identifier for the subject 102 and/or the test (e.g., a date and/or time of the test). In some examples, the distance calculation circuitry 330 causes the mobile device interface circuitry 310 to relay the test results database 280 to the mobile device 110. In some examples, the distance calculation circuitry 330 is instantiated by programmable circuitry executing measurement processing instructions and/or configured to perform operations such as those represented by the flowchart of FIG. 6.

The confidence determination circuitry 340 of FIG. 3 determines a confidence score to attribute to the determined total distance that the subject 102 traveled based on the signal strength data. In some examples, the confidence determination circuitry 340 determines the confidence score based on changes in the signal strength data. For example, when a range in the signal strength throughout the test does not satisfy (e.g., is less than, is less than or equal to) a range threshold (e.g., 10 dB, 15 dB, etc.), the confidence determination circuitry 340 can determine that the mobile device 110 did not measure reliable signals (e.g., measured radio frequency jitter), and therefore the confidence determination circuitry 340 attributes a confidence score of 0 to the test results. Further, the confidence determination circuitry 340 can cause the mobile device interface circuitry 310 to transmit the confidence score to the mobile device 110 with suggestions for addressing the source of the low confidence. For example, the confidence determination circuitry 340 can suggest that a new testing environment be utilized and/or that the subject 102 verify that the beacons are functioning properly. In some examples, the confidence determination circuitry 340 determines the confidence score based on a standard deviation of identified local maximum and/or minimum signal strengths and/or a difference between the signal strength range for the track defined by the stationary beacons 132, 134 and the signal strength range received from the mobile beacon 104 during complete traversals between the end markers 106, 108.

In some examples, confidence determination circuitry 340 determines a quantity of rests that the subject took based on the signal strength data. In some examples, the confidence determination circuitry 340 identifies a quantity of rests reported by the subject 102 via the mobile device 110. In some examples, the external device 120 compares the indicated quantity of rests to the determined quantity of rests. When the external device 120 determines that the indicated quantity of rests differs from the determined quantity of rests by at least a threshold amount (e.g., 1, 2, etc.), the external device 120 can cause the confidence score associated with the particular test to be reduced.

In some examples, the confidence determination circuitry 340 compares the determined confidence score to one or more threshold(s). For example, when the confidence score satisfies (e.g., is greater than, is greater than or equal to) a first threshold, the confidence determination circuitry 340 determines that the test results can be viewed and/or assessed with high confidence. In other words, the confidence determination circuitry 340 approves the results. In some examples, when the confidence score does not satisfy (e.g., is less than or equal to, is less than) the first threshold, the confidence determination circuitry 340 determines that the results may be less accurate or exact. In some examples, when the confidence score does not satisfy a second threshold lower than the first threshold, the confidence determination circuitry 340 recommends that the results be discarded or ignored. In some examples, when the confidence score does not satisfy a threshold, the confidence determination circuitry 340 causes the mobile device interface circuitry 310 to transmit a signal to the mobile device 110. In such examples, when the external device interface circuitry 260 (FIG. 2) receives the signal, the guidance circuitry 220 (FIG. 2) can cause the user interface circuitry 210 (FIG. 2) to prompt the subject 102 to change a location of the track 112, check and/or change the batteries in the mobile beacon 104, and/or submit the test results with an indication of the low confidence score.

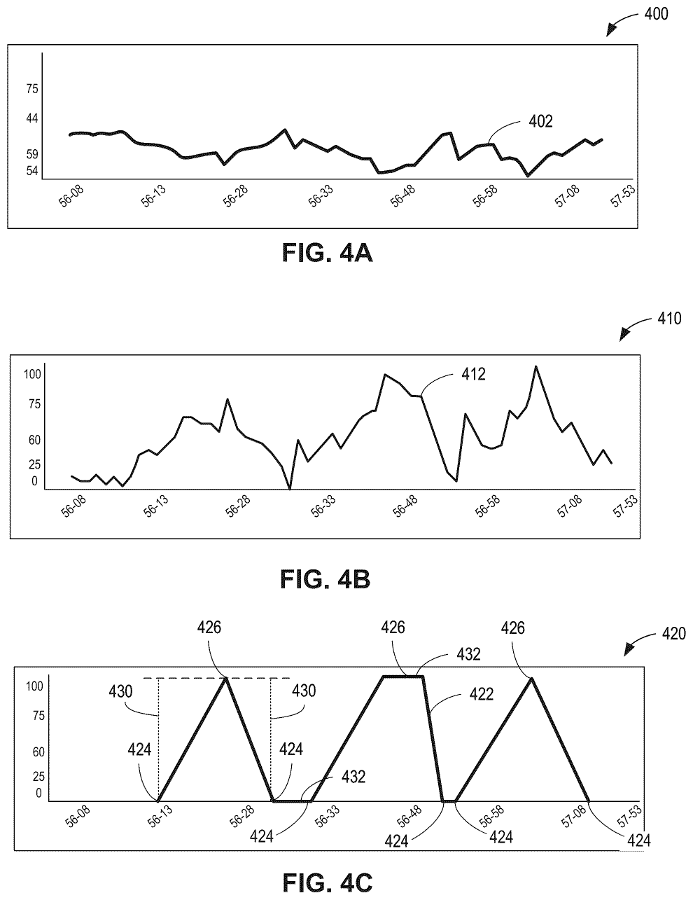

FIG. 4A illustrates a first graph 400 of example raw signal strength data 402 received by the mobile device 110 from the mobile beacon 104 during the six-minute walk test. Specifically, the raw signal strength data 402 corresponds to periodic strengths of signals that the signal receiver circuitry 230 receives from the mobile beacon 104. Accordingly, the signal measurement circuitry 250 measures the strength of the signals from the mobile beacon 104 and records the strengths over time, as shown in the first graph 400. The mobile device 110 transmits the raw signal strength data 402 to the external device(s) 120 for processing.

FIG. 4B illustrates a second graph 410 of example partially processed signal strength data 412. Specifically, the measurement processing circuitry 320 normalizes the raw signal strength data 402 of FIG. 4A to obtain the partially processed signal strength data 412.

FIG. 4C illustrates a third graph 420 of example further processed signal strength data 422. Specifically, the measurement processing circuitry 320 removes outliers in the partially processed signal strength data 412 of FIG. 4B to obtain the further processed signal strength data 422. In turn, the distance calculation circuitry 330 can analyze the further processed signal strength data 422 to determine the total distance that the subject traveled during the six-minute walk test. For example, the distance calculation circuitry 330 can identify local minimum strengths 424 indicative of the subject 102 being at the first end marker 106. Further, the distance calculation circuitry 330 can identify local maximum strengths 426 indicative of the subject 102 being at the second stationary beacon 134. Although the local minimum strengths 424 and the local maximums 426 of the illustrated example of FIG. 4C are approximately equivalent, it should be understood that minimums and maximums in the signal strength will vary as a result of slight variations in the position of the mobile beacon 104, a frequency at which signals are received from the mobile beacon 104, and/or signal interference in the test environment.

In some examples, the distance calculation circuitry 330 identifies the local minimum 424 in response to an increase in the signal strength satisfying (e.g., being greater than, being greater than or equal to) a threshold increase (e.g., a 5 dB increase) when the signal strength had been decreasing for a certain number of datapoints prior to satisfying the threshold increase. In some examples, the distance calculation circuitry 330 identifies the local maximum 426 in response to a decrease in the signal strength satisfying (e.g., being less than, being less than or equal to) a threshold decrease (e.g., a 5 dB decrease) when the signal strength had been increasing for a certain number of datapoints prior to satisfying the threshold decrease. In some examples, the distance calculation circuitry 330 adjusts the threshold increase and/or the threshold decrease that corresponds to the local minimum 424 and the local maximum 426 based on an estimated speed of the subject 102. For example, the distance calculation circuitry 330 can estimate the speed of the subject based on a slope corresponding to the change in the signal strength time. In turn, the distance calculation circuitry 330 can increase the threshold when the slope does not satisfy (e.g., is less than, is less than or equal to) a first slope threshold and/or decrease the threshold when the slope satisfies (e.g., is greater than, is greater than or equal to) a second slope threshold.

In some examples, the distance calculation circuitry 330 determines an average local maximum based on the local maximum strengths 426 and an average local minimum based on the local minimum strengths 424. In such examples, the distance calculation circuitry 330 determines the respective maximum strengths 426 are indicative of the subject 102 being at the second stationary beacon 134 in response to the respective maximum strengths 426 being within a threshold range (e.g., within 15 dB) of the average local maximum and/or the maximum defined by the signal received from the second stationary beacon 134 at a start of the six-minute walk test. Similarly, the distance calculation circuitry 330 can determine the respective minimum strengths 424 are indicative of the subject 102 being at the first end marker 106 in response to the respective minimum strengths 424 being within a threshold range (e.g., within 15 dB) of the average local minimum and/or a minimum defined by the signal received from the first stationary beacon 132 at a start of the test.

In the illustrated example, the distance calculation circuitry 330 determines signal strength ranges 430 between sequential minimum strengths 424 and maximum strengths 426. The distance calculation circuitry 330 determines an average signal strength range for completed traversals between the end markers 106, 108 based on the signal strength ranges 430 associated with respective minimum and maximum strengths 424, 426 that the distance calculation circuitry 330 has determined are indicative of the subject 102 being at the respective end markers 106, 108. In response to identifying a partially completed traversal between the end markers 106, 108, the distance calculation circuitry 330 determines a signal strength range associated with the partially completed traversal and, in turn, determines a fraction or percentage of the traversal that was completed based on a ratio between the signal strength range associated with the partially completed traversal and the average signal strength range for completed traversals between the end markers 106, 108. The distance calculation circuitry 330 multiplies the determined quantity of traversals by the predetermined distance associated with the track 112 to obtain the total distance that the subject 102 traveled during the six-minute walk test.

In some examples, the distance calculation circuitry 330 determines that the subject 102 stopped for a period during the six-minute walk test in response to identifying a plateau 432 in the signal strength (e.g., where the further processed signal strength data 422 has a slope of approximately 0). In some examples, in response to the plateau 432 persisting for at least a threshold period of time (e.g., 5 seconds), the distance calculation circuitry 330 determines that the plateau corresponds to a rest. Accordingly, the distance calculation circuitry 330 can determine a quantity of rests that the subject 102 took during the test based on a quantity of plateaus in the signal strength that are maintained for at least the threshold period of time. In some examples, the distance calculation circuitry 330 tracks a quantity of rests that the subject 102 took during the six-minute walk test. In some examples, the distance calculation circuitry 330 tracks an amount of time that the subject 102 was stopped during the six-minute walk test. Additionally, the distance calculation circuitry 330 can analyze the further processed signal strength data 422 to determine other statistics based on the further processed signal strength data 422, such as a rate at which the subject 102 completed traversals, a difference or variance in the movement of the subject 102 over time, etc.

While an example manner of implementing the mobile device 110 of FIGS. 1A-1D is illustrated in FIG. 2, one or more of the elements, processes, and/or devices illustrated in FIG. 2 may be combined, divided, re-arranged, omitted, eliminated, and/or implemented in any other way. Further, the user interface circuitry 210, the guidance circuitry 220, the signal receiver circuitry 230, the signal selection circuitry 240, the signal measurement circuitry 250, the external device interface circuitry 260, the configuration database 270, the test results database 280, and/or, more generally, the example mobile device 110 of FIGS. 1A-1D, may be implemented by hardware alone or by hardware in combination with software and/or firmware. Thus, for example, any of the user interface circuitry 210, the guidance circuitry 220, the signal receiver circuitry 230, the signal selection circuitry 240, the signal measurement circuitry 250, the external device interface circuitry 260, the configuration database 270, the test results database 280, and/or, more generally, the example mobile device 110, could be implemented by programmable circuitry, analog circuit(s), digital circuit(s), logic circuit(s), programmable processor(s), programmable microcontroller(s), graphics processing unit(s) (GPU(s)), digital signal processor(s) (DSP(s)), application specific integrated circuit(s) (ASIC(s)), programmable logic device(s) (PLD(s)), and/or field programmable logic device(s) (FPLD(s)) such as Field Programmable Gate Arrays (FPGAs). Further still, the example mobile device 110 of FIGS. 1A-1D may include one or more elements, processes, and/or devices in addition to, or instead of, those illustrated in FIG. 2, and/or may include more than one of any or all of the illustrated elements, processes, and devices.

While an example manner of implementing the external device(s) 120 of FIGS. 1A-1D is illustrated in FIG. 3, one or more of the elements, processes, and/or devices illustrated in FIG. 3 may be combined, divided, re-arranged, omitted, eliminated, and/or implemented in any other way. Further, the mobile device interface circuitry 310, the measurement processing circuitry 320, the distance calculation circuitry 330, the confidence determination circuitry 340, the configuration database 270, the test results database 280, and/or, more generally, the example external device(s) 120 of FIGS. 1A-1D, may be implemented by hardware alone or by hardware in combination with software and/or firmware. Thus, for example, any of mobile device interface circuitry 310, the measurement processing circuitry 320, the distance calculation circuitry 330, the confidence determination circuitry 340, the configuration database 270, the test results database 280, and/or, more generally, the example external device(s) 120, could be implemented by programmable circuitry, analog circuit(s), digital circuit(s), logic circuit(s), programmable processor(s), programmable microcontroller(s), GPU(s), DSP(s), ASIC(s), PLD(s), and/or FPLD(s) such as FPGAs. Further still, the example external device(s) 120 of FIGS. 1A-1D may include one or more elements, processes, and/or devices in addition to, or instead of, those illustrated in FIG. 3, and/or may include more than one of any or all of the illustrated elements, processes, and devices.

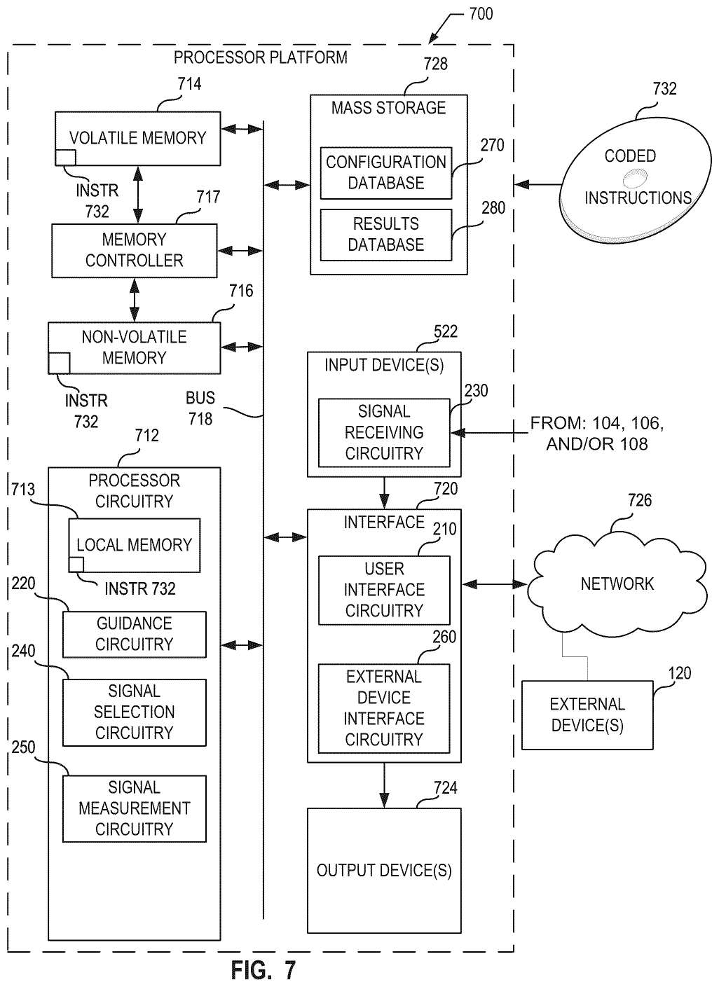

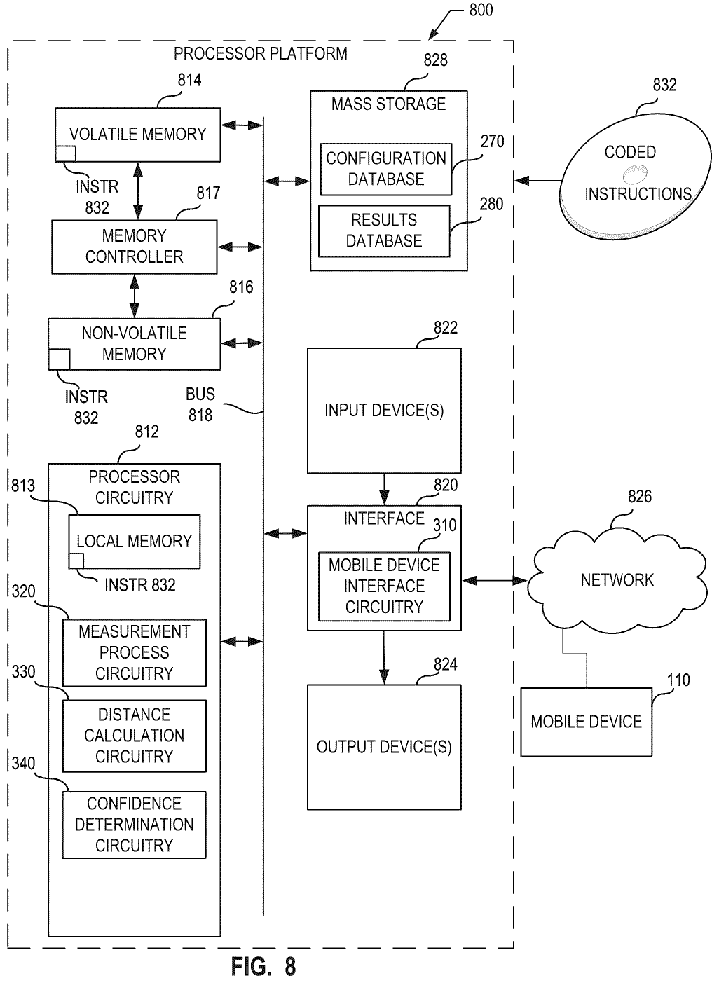

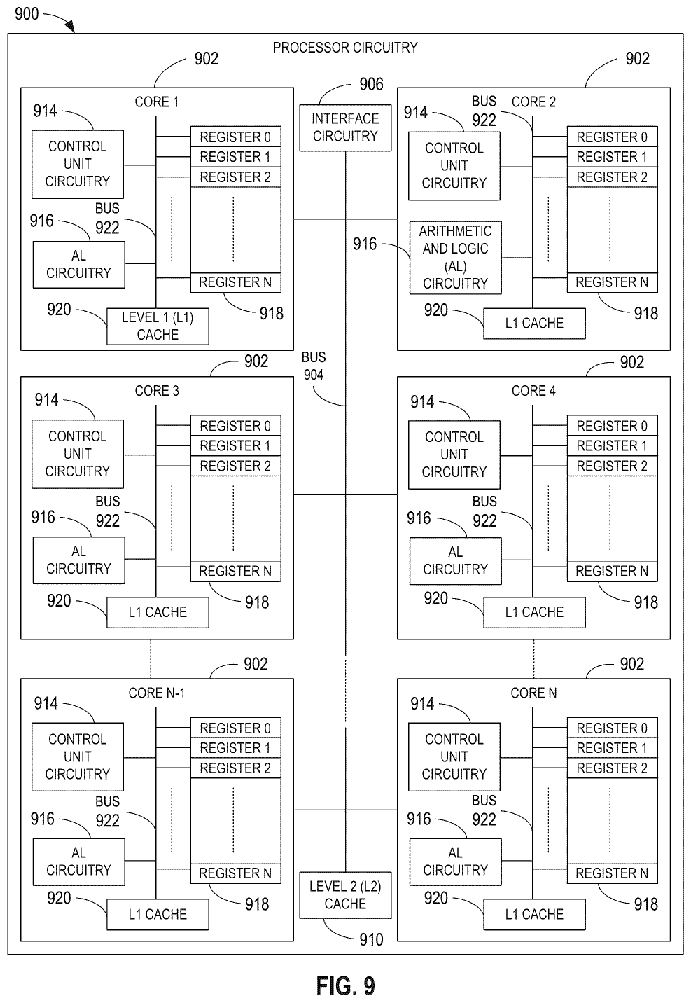

A flowchart representative of example machine readable instructions, which may be executed to configure programmable circuitry to implement the mobile device 110 of FIG. 2, is shown in FIG. 5. The machine readable instructions may be one or more executable programs or portion(s) of an executable program for execution by programmable circuitry, such as the programmable circuitry 712 shown in the example processor platform 700 discussed below in connection with FIG. 7 and/or the example programmable circuitry discussed below in connection with FIGS. 9 and/or 10. A flowchart representative of example machine readable instructions, which may be executed to configure programmable circuitry to implement the external device(s) 120 of FIG. 3, is shown in FIG. 6. The machine readable instructions may be one or more executable programs or portion(s) of an executable program for execution by programmable circuitry, such as the programmable circuitry 812 shown in the example processor platform 800 discussed below in connection with FIG. 8 and/or the example programmable circuitry discussed below in connection with FIGS. 9 and/or 10. The programs may be embodied in software stored on one or more non-transitory computer readable storage media such as a compact disk (CD), a floppy disk, a hard disk drive (HDD), a solid-state drive (SSD), a digital versatile disk (DVD), a Blu-ray disk, a volatile memory (e.g., Random Access Memory (RAM) of any type, etc.), or a non-volatile memory (e.g., electrically erasable programmable read-only memory (EEPROM), FLASH memory, an HDD, an SSD, etc.) associated with programmable circuitry located in one or more hardware devices, but the entire program and/or parts thereof could alternatively be executed by one or more hardware devices other than the programmable circuitry and/or embodied in firmware or dedicated hardware. The machine readable instructions may be distributed across multiple hardware devices and/or executed by two or more hardware devices (e.g., a server and a client hardware device). For example, the client hardware device may be implemented by an endpoint client hardware device (e.g., a hardware device associated with a user) or an intermediate client hardware device (e.g., a radio access network (RAN)) gateway that may facilitate communication between a server and an endpoint client hardware device). Similarly, the non-transitory computer readable storage media may include one or more mediums located in one or more hardware devices. Further, although the example programs are described with reference to the flowcharts illustrated in FIGS. 5-6, many other methods of implementing the example mobile device 110 and the example external device(s) 120 may alternatively be used. For example, the order of execution of the blocks may be changed, and/or some of the blocks described may be changed, eliminated, or combined. Additionally or alternatively, any or all of the blocks may be implemented by one or more hardware circuits (e.g., programmable circuitry, discrete and/or integrated analog and/or digital circuitry, an FPGA, an ASIC, a comparator, an operational-amplifier (op-amp), a logic circuit, etc.) structured to perform the corresponding operation without executing software or firmware. The programmable circuitry may be distributed in different network locations and/or local to one or more hardware devices (e.g., a single-core processor (e.g., a single core central processor unit (CPU)), a multi-core processor (e.g., a multi-core CPU, an XPU, etc.) in a single machine, multiple processors distributed across multiple servers of a server rack, multiple processors distributed across one or more server racks, a CPU and/or a FPGA located in the same package (e.g., the same integrated circuit (IC) package or in two or more separate housings, etc.).

The machine readable instructions described herein may be stored in one or more of a compressed format, an encrypted format, a fragmented format, a compiled format, an executable format, a packaged format, etc. Machine readable instructions as described herein may be stored as data or a data structure (e.g., as portions of instructions, code, representations of code, etc.) that may be utilized to create, manufacture, and/or produce machine executable instructions. For example, the machine readable instructions may be fragmented and stored on one or more storage devices and/or computing devices (e.g., servers) located at the same or different locations of a network or collection of networks (e.g., in the cloud, in edge devices, etc.). The machine readable instructions may require one or more of installation, modification, adaptation, updating, combining, supplementing, configuring, decryption, decompression, unpacking, distribution, reassignment, compilation, etc., in order to make them directly readable, interpretable, and/or executable by a computing device and/or other machine. For example, the machine readable instructions may be stored in multiple parts, which are individually compressed, encrypted, and/or stored on separate computing devices, wherein the parts when decrypted, decompressed, and/or combined form a set of machine executable instructions that implement one or more operations that may together form a program such as that described herein.

In another example, the machine readable instructions may be stored in a state in which they may be read by programmable circuitry, but require addition of a library (e.g., a dynamic link library (DLL)), a software development kit (SDK), an application programming interface (API), etc., in order to execute the machine readable instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, machine readable media, as used herein, may include machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example operations of FIGS. 5-6 may be implemented using executable instructions (e.g., computer and/or machine readable instructions) stored on one or more non-transitory computer and/or machine readable media such as optical storage devices, magnetic storage devices, an HDD, a flash memory, a read-only memory (ROM), a CD, a DVD, a cache, a RAM of any type, a register, and/or any other storage device or storage disk in which information is stored for any duration (e.g., for extended time periods, permanently, for brief instances, for temporarily buffering, and/or for caching of the information). As used herein, the terms non-transitory computer readable medium, non-transitory computer readable storage medium, non-transitory machine readable medium, and non-transitory machine readable storage medium are expressly defined to include any type of computer readable storage device and/or storage disk and to exclude propagating signals and to exclude transmission media. As used herein, the terms “computer readable storage device” and “machine readable storage device” are defined to include any physical (mechanical and/or electrical) structure to store information, but to exclude propagating signals and to exclude transmission media. Examples of computer readable storage devices and machine readable storage devices include random access memory of any type, read only memory of any type, solid state memory, flash memory, optical discs, magnetic disks, disk drives, and/or redundant array of independent disks (RAID) systems. As used herein, the term “device” refers to physical structure such as mechanical and/or electrical equipment, hardware, and/or circuitry that may or may not be configured by computer readable instructions, machine readable instructions, etc., and/or manufactured to execute computer readable instructions, machine readable instructions, etc.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements or method actions may be implemented by, e.g., the same entity or object. Additionally, although individual features may be included in different examples or claims, these may possibly be combined, and the inclusion in different examples or claims does not imply that a combination of features is not feasible and/or advantageous.

FIG. 5 is a flowchart representative of example machine readable instructions and/or example operations 500 that may be executed and/or instantiated by programmable circuitry to utilize signal strengths from Bluetooth beacons to obtain information indicative of a distance that a subject traveled during a six-minute walk test. The machine readable instructions and/or the operations 500 of FIG. 5 begin at block 502, at which the mobile device 110 (FIGS. 1A-1D and 2) receives signals (e.g., Bluetooth signals) from the first stationary beacon 132 (FIG. 1B) and the second stationary beacon 134 (FIG. 1B). For example, the signal receiver circuitry 230 (FIG. 2) can receive the signals from the end markers 106, 108. In some examples, the signal selection circuitry 240 (FIG. 2) identifies the signals associated with the stationary beacons 132, 134 based on identifiers carried by the signals. In such examples, the respective identifiers carried by signals transmitted by the first and second stationary beacons 132, 134 are stored in the configuration database 270. In some examples, the operations 500 proceed without receiving the stationary beacon signals (e.g., without executing block 502).

At block 504, the mobile device 110 determines an expected strength range to be received from the mobile beacon 104 during the six-minute walk test. For example, the signal selection circuitry 240 determines the expected strength range based on the respective strengths of the signals that the signal receiver circuitry 230 received from the stationary beacons 132, 134. In some examples, the signal selection circuitry 240 stores the expected strength range via the configuration database 270. In some examples, the operations 500 proceed without determining an expected signal strength range for the mobile beacon 104 (e.g., without executing block 504).

At block 506, the mobile device 110 pings the mobile beacon 104. For example, the signal selection circuitry 240 can transmit a query signal at a first frequency (e.g., every 0.25 seconds, every 0.5 seconds, etc.).

At block 508, the mobile device 110 receives a signal from the mobile beacon 104. For example, the signal receiver circuitry 230 can receive the signal from the mobile beacon 104. In some examples, the query signal causes the mobile beacon 104 to transmit the signal. In some examples, the mobile beacon 104 transmits the signal on its own at a second frequency less than the first frequency. In some examples, the signal selection circuitry 240 determines which signal originated from the mobile beacon 104 based on an identifier carried by the signal and/or the expected strength range. In some examples, the signal selection circuitry 240 looks up the identifier associated with the mobile beacon 104 via the configuration database 270.

At block 510, the mobile device 110 measures a strength (e.g., an attenuation) of the signal received from the mobile beacon 104. For example, the signal measurement circuitry 250 (FIG. 2) can measure the strength of the signals received from the mobile beacon 104.

At block 512, the mobile device 110 records the strength with a timestamp. For example, the signal measurement circuitry 250 can store a data point indicative of the measured strength and the time stamp in the test results database 280 (FIG. 2). In some examples, the signal measurement circuitry 250 stores the data points in a graph format (e.g., the first graph 300 (FIG. 3)).

At block 514, the mobile device 110 determines whether motion of the subject 102 has been detected. For example, the signal measurement circuitry 250 can determine the subject 102 is moving in response to the strength of the signal from the mobile beacon 104 changing over time. In response to motion being detected, the operations 500 skip to block 518. Otherwise, in response to motion not being detected, the operations 500 proceed to block 516.

At block 516, the mobile device 110 prompts the subject 102 to troubleshoot the test setup. For example, in response to not detecting a change in the strength of the signal from the mobile beacon 104, the signal measurement circuitry 250 can cause the user interface circuitry 210 to present an audible and/or visual alert to the subject 102. In some examples, the alert prompts the subject 102 to ensure that the beacons 104, 132, 134 are powered on. In some examples, the user interface circuitry 210 presents contact information that the subject 102 can utilize to help address the issue.

At block 518, the mobile device 110 determines whether the six-minute walk test is complete. For example, the guidance circuitry 220 (FIG. 2) can identify whether the six-minute period for walking has expired. In response to the six-minute walk test being complete (e.g., in response to the six-minute period expiring), the operations 500 proceed to block 520. Otherwise, the operations 500 return to block 506.

At block 520, the mobile device 110 transmits the raw signal strength data to the external device(s) 120. For example, the external device interface circuitry 260 (FIG. 2) can transmit the raw signal strength data to the external device(s) 120 (FIGS. 1A-1D and 3) via the network 122 (FIGS. 1A-1D). In some examples, the external device interface circuitry 260 transmits the configuration database 270 and/or the test results database 280 to the external device(s) 120.

At block 522, the mobile device 110 determines whether the test results have been received. For example, the external device interface circuitry 260 can determine whether the total travel distance and/or a confidence score has been received from the external device(s) 120. In response to the test results not yet being received, the operations 500 repeat at block 522. Otherwise, in response to the test results being received, the operations 500 proceed to block 524.

At block 524, the mobile device 110 presents the test results. For example, in response to receiving the results from the external device(s) 120, the external device interface circuitry 260 can cause the user interface circuitry 210 to present the results.

FIG. 6 is a flowchart representative of example machine readable instructions and/or example operations 600 that may be executed and/or instantiated by programmable circuitry to determine a distance that a subject traveled during a six-minute walk test based on signals transmitted by Bluetooth beacons. The machine readable instructions and/or the operations 600 of FIG. 6 begin at block 602, at which the external device(s) 120 (FIGS. 1A-1D and 3) determine whether raw signal strength data has been received. For example, the mobile device interface circuitry 310 (FIG. 3) can determine whether the raw signal strength data associated with a six-minute walk test completed by the subject 102 (FIGS. 1A-1D) has been received. In response to the raw signal strength data not yet being received, the operations 600 repeat at block 602. Otherwise, in response to the raw signal strength data being received, the operations 600 proceed to block 604.

At block 604, the external device(s) 120 normalize the signal strength data. For example, the measurement processing circuitry 320 (FIG. 3) can normalize the signal data stored in the test results database 280 to obtain partially processed strength data (e.g., the partially processed signal data 410 (FIG. 4B)).

At block 606, the external device(s) 120 remove outliers in the signal strength data. For example, the measurement processing circuitry 320 can remove outliers from the normalized data to obtain processed signal data (e.g., the further processed strength data 420 (FIG. 4C)). In some examples, the measurement processing circuitry 320 applies a 5-point moving average to the partially processed strength data to remove the outliers.

At block 608, the external device(s) 120 identify completed traversals. For example, the distance calculation circuitry 330 (FIG. 3) can identify a quantity of traversals (e.g., trips) that the subject 102 completed between the end markers 106, 108 based on changes in the slope of the processed signal data and local maximum and minimum strengths in the processed signal data.

At block 610, the external device(s) 120 determine whether any traversals by the subject 102 between the end markers 106, 108 were not completed. In some examples, the distance calculation circuitry 330 determines a traversal between the end markers 106, 108 was not fully completed in response to an identified local maximum or minimum strength not being within a threshold range (e.g., 15 dB) of the respective maximum or minimum defined by signal strength range associated with the track and/or the average local maximum or minimum signal strength measured during the test. In response to any traversals not being fully completed, the operations 600 proceed to block 612. Otherwise, the operations 600 skip to block 614.

At block 612, the external device(s) 120 determine a percentage completion for the traversal(s) that were not fully completed by the subject 102 during the six-minute walk test. In some examples, the distance calculation circuitry 330 determines a signal strength range associated with the partially completed traversal. In such examples, the distance calculation circuitry 330 determines a fraction or percentage of the traversal that was completed based on a ratio between the signal strength range associated with the partially completed traversal and the average signal strength range for completed traversals between the end markers 106, 108.

At block 614, the external device(s) 120 collate the completed and partially completed traversals. For example, the distance calculation circuitry 330 can sum the fully completed and the partially completed traversals to obtain a total quantity of traversals that the subject 102 made between the end markers 106, 108 during the six-minute walk test.

At block 616, the external device(s) 120 multiply the summed quantity of traversals by a predetermined distance associated with the track 112. For example, the distance calculation circuitry 330 can multiply the quantity of traversals by the length of the track 112. In some examples, the distance calculation circuitry 330 identifies the length of the track via the configuration database 270.

At block 618, the external device(s) 120 determine a confidence score associated with the test results. For example, the confidence determination circuitry 340 can determine the confidence score based the signal strength data. In some examples, the confidence determination circuitry 340 determines a recommendation for the subject 102 based on the signal strength data.

At block 620, the external device(s) 120 output the test results. For example, the mobile device interface circuitry 310 can transmit the test results to the mobile device 110.

FIG. 7 is a block diagram of an example processor platform 700 structured to execute and/or instantiate the machine readable instructions and/or the operations of FIG. 5 to implement the mobile device 110 of FIGS. 1A-1D and 2. The processor platform 700 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset (e.g., an augmented reality (AR) headset, a virtual reality (VR) headset, etc.) or other wearable device, or any other type of computing device.

The processor platform 700 of the illustrated example includes programmable circuitry 712. The programmable circuitry 712 of the illustrated example is hardware. For example, the programmable circuitry 712 can be implemented by one or more integrated circuits, logic circuits, FPGAs, microprocessors, CPUs, GPUs, DSPs, and/or microcontrollers from any desired family or manufacturer. The programmable circuitry 712 may be implemented by one or more semiconductor based (e.g., silicon based) devices. In this example, the programmable circuitry 712 implements the guidance circuitry 220, the signal selection circuitry 240, and the signal measurement circuitry 250.

The programmable circuitry 712 of the illustrated example includes a local memory 713 (e.g., a cache, registers, etc.). The programmable circuitry 712 of the illustrated example is in communication with a main memory including a volatile memory 714 and a non-volatile memory 716 by a bus 718. The volatile memory 714 may be implemented by Synchronous Dynamic Random Access Memory (SDRAM), Dynamic Random Access Memory (DRAM), RAMBUS® Dynamic Random Access Memory (RDRAM®), and/or any other type of RAM device. The non-volatile memory 716 may be implemented by flash memory and/or any other desired type of memory device. Access to the main memory 714, 716 of the illustrated example is controlled by a memory controller 717.

The processor platform 700 of the illustrated example also includes interface circuitry 720. The interface circuitry 720 may be implemented by hardware in accordance with any type of interface standard, such as an Ethernet interface, a universal serial bus (USB) interface, a Bluetooth® interface, a near field communication (NFC) interface, a Peripheral Component Interconnect (PCI) interface, and/or a Peripheral Component Interconnect Express (PCIe) interface.

In the illustrated example, one or more input devices 722 are connected to the interface circuitry 720. The input device(s) 722 permit(s) a user to enter data and/or commands into the programmable circuitry 712. The input device(s) 722 can be implemented by, for example, a Bluetooth signal receiver, an audio sensor, a microphone, a camera (still or video), a keyboard, a button, a mouse, a touchscreen, a track-pad, a trackball, an isopoint device, and/or a voice recognition system. In this example, the input device(s) 722 implement the signal receiver circuitry 230.

One or more output devices 724 are also connected to the interface circuitry 720 of the illustrated example. The output device(s) 724 can be implemented, for example, by a transmitter, display devices (e.g., a light emitting diode (LED), an organic light emitting diode (OLED), a liquid crystal display (LCD), a cathode ray tube (CRT) display, an in-place switching (IPS) display, a touchscreen, etc.), a tactile output device, a printer, and/or speaker. The interface circuitry 720 of the illustrated example, thus, typically includes a graphics driver card, a graphics driver chip, and/or graphics programmable circuitry such as a GPU.

The interface circuitry 720 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 726. In this example, the external device(s) 120 are in communication with the network 726. The communication can be by, for example, an Ethernet connection, a digital subscriber line (DSL) connection, a telephone line connection, a coaxial cable system, a satellite system, a line-of-site wireless system, a cellular telephone system, an optical connection, etc. In this example, the interface circuitry 720 implements the user interface circuitry 210 and the external device interface circuitry 260.

The processor platform 700 of the illustrated example also includes one or more mass storage devices 728 to store software and/or data. Examples of such mass storage devices 728 include magnetic storage devices, optical storage devices, floppy disk drives, HDDs, CDs, Blu-ray disk drives, redundant array of independent disks (RAID) systems, solid state storage devices such as flash memory devices and/or SSDs, and DVD drives.

The machine readable instructions 732, which may be implemented by the machine readable instructions of FIG. 5, may be stored in the mass storage device 728, in the volatile memory 714, in the non-volatile memory 716, and/or on a removable non-transitory computer readable storage medium such as a CD or DVD. In this example, the mass storage device 728 implements the configuration database 270 and the test results database 280.

FIG. 8 is a block diagram of an example processor platform 800 structured to execute and/or instantiate the machine readable instructions and/or the operations of FIG. 6 to implement the external device(s) 120 of FIGS. 1A-1D and 3. The processor platform 800 can be, for example, a server, a personal computer, a workstation, a self-learning machine (e.g., a neural network), a mobile device (e.g., a cell phone, a smart phone, a tablet such as an iPad™), a PDA, an Internet appliance, a DVD player, a CD player, a digital video recorder, a Blu-ray player, a gaming console, a personal video recorder, a set top box, a headset (e.g., an AR headset, a VR headset, etc.) or other wearable device, or any other type of computing device.

The processor platform 800 of the illustrated example includes programmable circuitry 812. The programmable circuitry 812 of the illustrated example is hardware. For example, the programmable circuitry 812 can be implemented by one or more integrated circuits, logic circuits, FPGAs, microprocessors, CPUs, GPUs, DSPs, and/or microcontrollers from any desired family or manufacturer. The programmable circuitry 812 may be implemented by one or more semiconductor based (e.g., silicon based) devices. In this example, the programmable circuitry 812 implements the measurement processing circuitry 320, the distance calculation circuitry 330, and the confidence determination circuitry 340.

The programmable circuitry 812 of the illustrated example includes a local memory 813 (e.g., a cache, registers, etc.). The programmable circuitry 812 of the illustrated example is in communication with a main memory including a volatile memory 814 and a non-volatile memory 816 by a bus 818. The volatile memory 814 may be implemented by SDRAM, DRAM, RDRAM®, and/or any other type of RAM device. The non-volatile memory 816 may be implemented by flash memory and/or any other desired type of memory device. Access to the main memory 814, 816 of the illustrated example is controlled by a memory controller 817.

The processor platform 800 of the illustrated example also includes interface circuitry 820. The interface circuitry 820 may be implemented by hardware in accordance with any type of interface standard, such as an Ethernet interface, a USB interface, a Bluetooth® interface, an NFC interface, a PCI interface, and/or a PCIe interface.

In the illustrated example, one or more input devices 822 are connected to the interface circuitry 820. The input device(s) 822 permit(s) a user to enter data and/or commands into the programmable circuitry 812. The input device(s) 822 can be implemented by, for example, a Bluetooth signal receiver, an audio sensor, a microphone, a camera (still or video), a keyboard, a button, a mouse, a touchscreen, a track-pad, a trackball, an isopoint device, and/or a voice recognition system.

One or more output devices 824 are also connected to the interface circuitry 820 of the illustrated example. The output device(s) 824 can be implemented, for example, by a transmitter, display devices (e.g., an LED, an OLED, an LCD, a CRT display, an IPS display, a touchscreen, etc.), a tactile output device, a printer, and/or speaker. The interface circuitry 820 of the illustrated example, thus, typically includes a graphics driver card, a graphics driver chip, and/or graphics programmable circuitry such as a GPU.

The interface circuitry 820 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 826. In this example, the mobile device 110 is in communication with the network 826. The communication can be by, for example, an Ethernet connection, a DSL connection, a telephone line connection, a coaxial cable system, a satellite system, a line-of-site wireless system, a cellular telephone system, an optical connection, etc. In this example, the interface circuitry 820 implements the mobile device interface circuitry 310.

The processor platform 800 of the illustrated example also includes one or more mass storage devices 828 to store software and/or data. Examples of such mass storage devices 828 include magnetic storage devices, optical storage devices, floppy disk drives, HDDs, CDs, Blu-ray disk drives, RAID systems, solid state storage devices such as flash memory devices and/or SSDs, and DVD drives.

The machine readable instructions 832, which may be implemented by the machine readable instructions of FIG. 6, may be stored in the mass storage device 828, in the volatile memory 814, in the non-volatile memory 816, and/or on a removable non-transitory computer readable storage medium such as a CD or DVD. In this example, the mass storage device 828 implements the configuration database 270 and the test results database 280.

FIG. 9 is a block diagram of an example implementation of the programmable circuitry 712 of FIG. 7 and/or the programmable circuitry 812 of FIG. 8. In this example, the programmable circuitry 712 of FIG. 7 and/or the programmable circuitry 812 of FIG. 8 is implemented by a microprocessor 900. For example, the microprocessor 900 may be a general purpose microprocessor (e.g., general purpose microprogrammable circuitry). The microprocessor 900 executes some or all of the machine readable instructions of the flowcharts of FIGS. 5-6 to effectively instantiate the mobile device 110 of FIG. 2 and/or the external device(s) 120 of FIG. 3 as logic circuits to perform the operations corresponding to those machine readable instructions. In some such examples, the mobile device 110 of FIG. 2 and/or the external device(s) 120 of FIG. 3 are instantiated by the hardware circuits of the microprocessor 900 in combination with the instructions. For example, the microprocessor 900 may be implemented by multi-core hardware circuitry such as a CPU, a DSP, a GPU, an XPU, etc. Although it may include any number of example cores 902 (e.g., 1 core), the microprocessor 900 of this example is a multi-core semiconductor device including N cores. The cores 902 of the microprocessor 900 may operate independently or may cooperate to execute machine readable instructions. For example, machine code corresponding to a firmware program, an embedded software program, or a software program may be executed by one of the cores 902 or may be executed by multiple ones of the cores 902 at the same or different times. In some examples, the machine code corresponding to the firmware program, the embedded software program, or the software program is split into threads and executed in parallel by two or more of the cores 902. The software program may correspond to a portion or all of the machine readable instructions and/or operations represented by the flowcharts of FIGS. 5-6.

The cores 902 may communicate by a first example bus 904. In some examples, the first bus 904 may be implemented by a communication bus to effectuate communication associated with one(s) of the cores 902. For example, the first bus 904 may be implemented by at least one of an Inter-Integrated Circuit (I2C) bus, a Serial Peripheral Interface (SPI) bus, a PCI bus, or a PCIe bus. Additionally or alternatively, the first bus 904 may be implemented by any other type of computing or electrical bus. The cores 902 may obtain data, instructions, and/or signals from one or more external devices by example interface circuitry 906. The cores 902 may output data, instructions, and/or signals to the one or more external devices by the interface circuitry 906. Although the cores 902 of this example include example local memory 920 (e.g., Level 1 (L1) cache that may be split into an L1 data cache and an L1 instruction cache), the microprocessor 900 also includes example shared memory 910 that may be shared by the cores (e.g., Level 2 (L2 cache)) for high-speed access to data and/or instructions. Data and/or instructions may be transferred (e.g., shared) by writing to and/or reading from the shared memory 910. The local memory 920 of each of the cores 902 and the shared memory 910 may be part of a hierarchy of storage devices including multiple levels of cache memory and the main memory (e.g., the main memory 714, 716 of FIG. 7, the main memory 814, 816 of FIG. 8). Typically, higher levels of memory in the hierarchy exhibit lower access time and have smaller storage capacity than lower levels of memory. Changes in the various levels of the cache hierarchy are managed (e.g., coordinated) by a cache coherency policy.

Each core 902 may be referred to as a CPU, DSP, GPU, etc., or any other type of hardware circuitry. Each core 902 includes control unit circuitry 914, arithmetic and logic (AL) circuitry (sometimes referred to as an ALU) 916, a plurality of registers 918, the local memory 920, and a second example bus 922. Other structures may be present. For example, each core 902 may include vector unit circuitry, single instruction multiple data (SIMD) unit circuitry, load/store unit (LSU) circuitry, branch/jump unit circuitry, floating-point unit (FPU) circuitry, etc. The control unit circuitry 914 includes semiconductor-based circuits structured to control (e.g., coordinate) data movement within the corresponding core 902. The AL circuitry 916 includes semiconductor-based circuits structured to perform one or more mathematic and/or logic operations on the data within the corresponding core 902. The AL circuitry 916 of some examples performs integer based operations. In other examples, the AL circuitry 916 also performs floating point operations. In yet other examples, the AL circuitry 916 may include first AL circuitry that performs integer based operations and second AL circuitry that performs floating point operations. In some examples, the AL circuitry 916 may be referred to as an Arithmetic Logic Unit (ALU). The registers 918 are semiconductor-based structures to store data and/or instructions such as results of one or more of the operations performed by the AL circuitry 916 of the corresponding core 902. For example, the registers 918 may include vector register(s), SIMD register(s), general purpose register(s), flag register(s), segment register(s), machine specific register(s), instruction pointer register(s), control register(s), debug register(s), memory management register(s), machine check register(s), etc. The registers 918 may be arranged in a bank as shown in FIG. 9. Alternatively, the registers 918 may be organized in any other arrangement, format, or structure including distributed throughout the core 902 to shorten access time. The second bus 922 may be implemented by at least one of an I2C bus, a SPI bus, a PCI bus, or a PCIe bus

Each core 902 and/or, more generally, the microprocessor 900 may include additional and/or alternate structures to those shown and described above. For example, one or more clock circuits, one or more power supplies, one or more power gates, one or more cache home agents (CHAs), one or more converged/common mesh stops (CMSs), one or more shifters (e.g., barrel shifter(s)) and/or other circuitry may be present. The microprocessor 900 is a semiconductor device fabricated to include many transistors interconnected to implement the structures described above in one or more integrated circuits (ICs) contained in one or more packages. The programmable circuitry may include and/or cooperate with one or more accelerators. In some examples, accelerators are implemented by logic circuitry to perform certain tasks more quickly and/or efficiently than can be done by a general purpose processor. Examples of accelerators include ASICs and FPGAs such as those discussed herein. A GPU or other programmable device can also be an accelerator. Accelerators may be on-board the programmable circuitry, in the same chip package as the programmable circuitry and/or in one or more separate packages from the programmable circuitry.

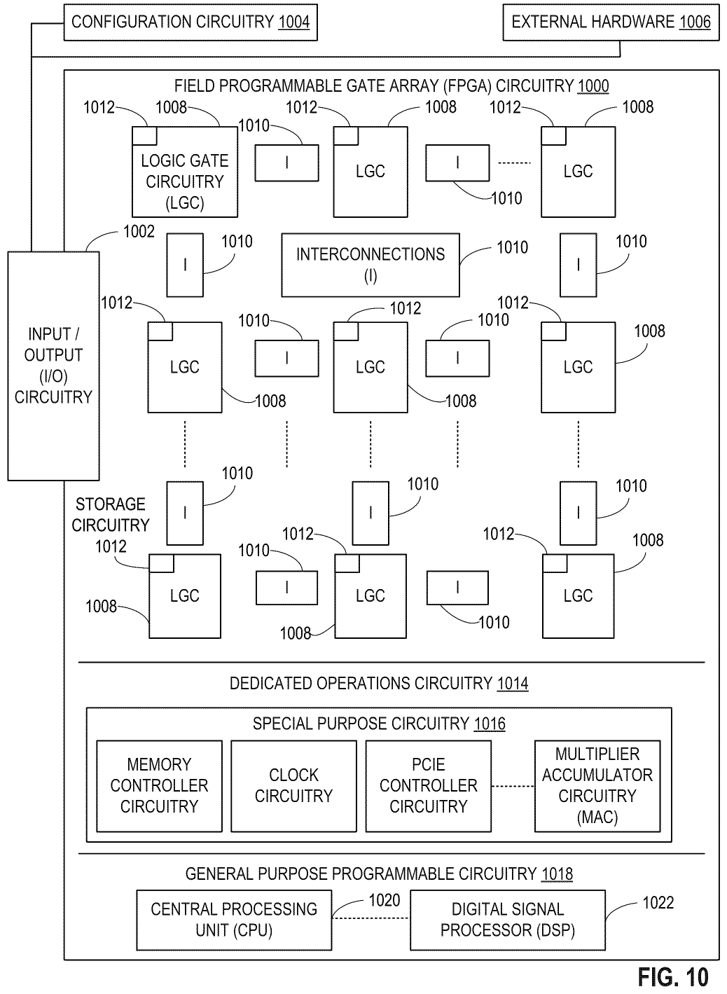

FIG. 10 is a block diagram of another example implementation of the programmable circuitry 712 of FIG. 7 and/or the programmable circuitry 812 of FIG. 8. In this example, the programmable circuitry 712 of FIG. 7 and/or the programmable circuitry 812 of FIG. 8 are implemented by FPGA circuitry 1000. For example, the FPGA circuitry 1000 may be implemented by an FPGA. The FPGA circuitry 1000 can be used, for example, to perform operations that could otherwise be performed by the example microprocessor 900 of FIG. 9 executing corresponding machine readable instructions. However, once configured, the FPGA circuitry 1000 instantiates the machine readable instructions in hardware and, thus, can often execute the operations faster than they could be performed by a general purpose microprocessor executing the corresponding software.

More specifically, in contrast to the microprocessor 900 of FIG. 9 described above (which is a general purpose device that may be programmed to execute some or all of the machine readable instructions represented by the flowcharts of FIGS. 5-6 but whose interconnections and logic circuitry are fixed once fabricated), the FPGA circuitry 1000 of the example of FIG. 10 includes interconnections and logic circuitry that may be configured and/or interconnected in different ways after fabrication to instantiate, for example, some or all of the machine readable instructions represented by the flowcharts of FIGS. 5-6. In particular, the FPGA circuitry 1000 may be thought of as an array of logic gates, interconnections, and switches. The switches can be programmed to change how the logic gates are interconnected by the interconnections, effectively forming one or more dedicated logic circuits (unless and until the FPGA circuitry 1000 is reprogrammed). The configured logic circuits enable the logic gates to cooperate in different ways to perform different operations on data received by input circuitry. Those operations may correspond to some or all of the software represented by the flowcharts of FIGS. 5-6. As such, the FPGA circuitry 1000 may be structured to effectively instantiate some or all of the machine readable instructions of the flowcharts of FIGS. 5-6 as dedicated logic circuits to perform the operations corresponding to those software instructions in a dedicated manner analogous to an ASIC. Therefore, the FPGA circuitry 1000 may perform the operations corresponding to the some or all of the machine readable instructions of FIGS. 5-6 faster than the general purpose microprocessor can execute the same.

In the example of FIG. 10, the FPGA circuitry 1000 is structured to be programmed (and/or reprogrammed one or more times) by an end user by a hardware description language (HDL) such as Verilog. The FPGA circuitry 1000 of FIG. 10, includes example input/output (I/O) circuitry 1002 to obtain and/or output data to/from example configuration circuitry 1004 and/or external hardware 1006. For example, the configuration circuitry 1004 may be implemented by interface circuitry that may obtain machine readable instructions to configure the FPGA circuitry 1000, or portion(s) thereof. In some such examples, the configuration circuitry 1004 may obtain the machine readable instructions from a user, a machine (e.g., hardware circuitry (e.g., programmed or dedicated circuitry) that may implement an Artificial Intelligence/Machine Learning (AI/ML) model to generate the instructions), etc. In some examples, the external hardware 1006 may be implemented by external hardware circuitry. For example, the external hardware 1006 may be implemented by the microprocessor 900 of FIG. 9. The FPGA circuitry 1000 also includes an array of example logic gate circuitry 1008, a plurality of example configurable interconnections 1010, and example storage circuitry 1012. The logic gate circuitry 1008 and the configurable interconnections 1010 are configurable to instantiate one or more operations that may correspond to at least some of the machine readable instructions of FIGS. 5-6 and/or other desired operations. The logic gate circuitry 1008 shown in FIG. 10 is fabricated in groups or blocks. Each block includes semiconductor-based electrical structures that may be configured into logic circuits. In some examples, the electrical structures include logic gates (e.g., And gates, Or gates, Nor gates, etc.) that provide basic building blocks for logic circuits. Electrically controllable switches (e.g., transistors) are present within each of the logic gate circuitry 1008 to enable configuration of the electrical structures and/or the logic gates to form circuits to perform desired operations. The logic gate circuitry 1008 may include other electrical structures such as look-up tables (LUTs), registers (e.g., flip-flops or latches), multiplexers, etc.

The configurable interconnections 1010 of the illustrated example are conductive pathways, traces, vias, or the like that may include electrically controllable switches (e.g., transistors) whose state can be changed by programming (e.g., using an HDL instruction language) to activate or deactivate one or more connections between one or more of the logic gate circuitry 1008 to program desired logic circuits.

The storage circuitry 1012 of the illustrated example is structured to store result(s) of the one or more of the operations performed by corresponding logic gates. The storage circuitry 1012 may be implemented by registers or the like. In the illustrated example, the storage circuitry 1012 is distributed amongst the logic gate circuitry 1008 to facilitate access and increase execution speed.

The example FPGA circuitry 1000 of FIG. 10 also includes example Dedicated Operations Circuitry 1014. In this example, the Dedicated Operations Circuitry 1014 includes special purpose circuitry 1016 that may be invoked to implement commonly used functions to avoid the need to program those functions in the field. Examples of such special purpose circuitry 1016 include memory (e.g., DRAM) controller circuitry, PCIe controller circuitry, clock circuitry, transceiver circuitry, memory, and multiplier-accumulator circuitry. Other types of special purpose circuitry may be present. In some examples, the FPGA circuitry 1000 may also include example general purpose programmable circuitry 1018 such as an example CPU 1020 and/or an example DSP 1022. Other general purpose programmable circuitry 1018 may additionally or alternatively be present such as a GPU, an XPU, etc., that can be programmed to perform other operations.

Although FIGS. 9 and 10 illustrate two example implementations of the programmable circuitry 712 of FIG. 7 and/or the programmable circuitry 812 of FIG. 8, many other approaches are contemplated. For example, as mentioned above, modern FPGA circuitry may include an on-board CPU, such as one or more of the example CPU 1020 of FIG. 10. Therefore, the programmable circuitry 712 of FIG. 7 and/or the programmable circuitry 812 of FIG. 8 may additionally be implemented by combining the example microprocessor 900 of FIG. 6 and the example FPGA circuitry 1000 of FIG. 10. In some such hybrid examples, a first portion of the machine readable instructions represented by the flowcharts of FIGS. 5-6 may be executed by one or more of the cores 902 of FIG. 9, a second portion of the machine readable instructions represented by the flowcharts of FIGS. 5-6 may be executed by the FPGA circuitry 1000 of FIG. 10, and/or a third portion of the machine readable instructions represented by the flowcharts of FIGS. 5-6 may be executed by an ASIC. It should be understood that some or all of the mobile device 110 of FIG. 2 and/or the external device(s) 120 of FIG. 3 may, thus, be instantiated at the same or different times. Some or all of the circuitry may be instantiated, for example, in one or more threads executing concurrently and/or in series. Moreover, in some examples, some or all of the mobile device 110 of FIG. 2 and/or the external device(s) 120 of FIG. 3 may be implemented within one or more virtual machines and/or containers executing on the microprocessor.

In some examples, the programmable circuitry 712 of FIG. 7 and/or the programmable circuitry 812 of FIG. 8 may be in one or more packages. For example, the microprocessor 900 of FIG. 9 and/or the FPGA circuitry 1000 of FIG. 10 may be in one or more packages. In some examples, an XPU may be implemented by the programmable circuitry 712 of FIG. 7 and/or the programmable circuitry 812 of FIG. 8, which may be in one or more packages. For example, the XPU may include a CPU in one package, a DSP in another package, a GPU in yet another package, and an FPGA in still yet another package.

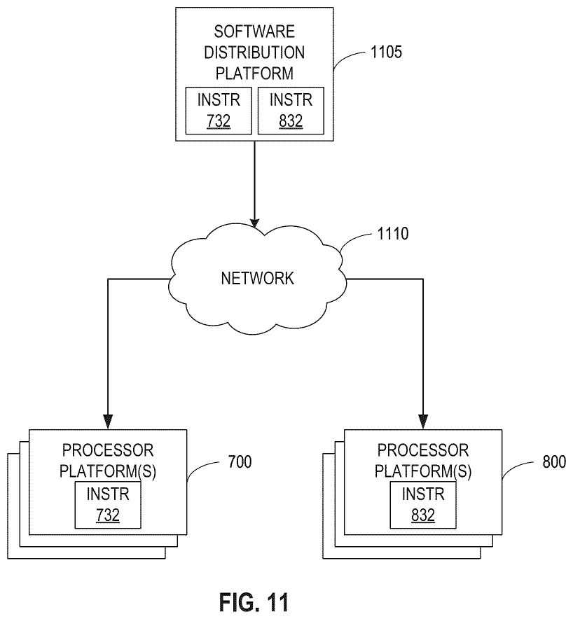

A block diagram illustrating an example software distribution platform 1105 to distribute software such as the example machine readable instructions 732 of FIG. 7 and/or the example machine readable instructions 832 of FIG. 8 to hardware devices owned and/or operated by third parties is illustrated in FIG. 11. The example software distribution platform 1105 may be implemented by any computer server, data facility, cloud service, etc., capable of storing and transmitting software to other computing devices. The third parties may be customers of the entity owning and/or operating the software distribution platform 1105. For example, the entity that owns and/or operates the software distribution platform 1105 may be a developer, a seller, and/or a licensor of software such as the example machine readable instructions 732 of FIG. 7 and/or the example machine readable instructions 832 of FIG. 8. The third parties may be consumers, users, retailers, OEMs, etc., who purchase and/or license the software for use and/or re-sale and/or sub-licensing. In the illustrated example, the software distribution platform 1105 includes one or more servers and one or more storage devices. The storage devices store the machine readable instructions 732 of FIG. 7 and/or the example machine readable instructions 832 of FIG. 8, which may correspond to the example machine readable instructions 500, 600 of FIGS. 5-6, as described above. The one or more servers of the example software distribution platform 1105 are in communication with an example network 1110, which may correspond to any one or more of the Internet and/or the example network 122, 726, 826 described above. In some examples, the one or more servers are responsive to requests to transmit the software to a requesting party as part of a commercial transaction. Payment for the delivery, sale, and/or license of the software may be handled by the one or more servers of the software distribution platform and/or by a third party payment entity. The servers enable purchasers and/or licensors to download the machine readable instructions 732 of FIG. 7 and/or the example machine readable instructions 832 of FIG. 8 from the software distribution platform 1105. For example, the software, which may correspond to the example machine readable instructions 500, 600 of FIGS. 5-6, may be downloaded to the example processor platform 1100, which is to execute the machine readable instructions 732 of FIG. 7 and/or the example machine readable instructions 832 of FIG. 8 to implement the mobile device 110 and/or the external device(s) 120. In some examples, one or more servers of the software distribution platform 1105 periodically offer, transmit, and/or force updates to the software (e.g., the example machine readable instructions 732 of FIG. 7 and/or the example machine readable instructions 832 of FIG. 8) to ensure improvements, patches, updates, etc., are distributed and applied to the software at the end user devices.

From the foregoing, it will be appreciated that example systems, methods, apparatus, and articles of manufacture have been disclosed that monitor distances traveled by subjects during six-minute walk tests performed in non-clinical settings. Further examples and combinations thereof include the following:

Example 1 includes a system comprising a mobile beacon to be carried by a subject during a six-minute walk test, interface circuitry, machine readable instructions, and programmable circuitry to at least one of instantiate or execute the machine readable instructions to determine a distance traveled by the subject during the six-minute walk test based on a strength of signals received from the mobile beacon.

Example 2 includes the system of example 1, wherein the programmable circuitry is to determine a quantity of traversals that the subject completed between a first end of a track associated with the six-minute walk test and a second end of the track based on a rate of change of the strength of the signals received from the mobile beacon.

Example 3 includes the system of example 1, wherein the programmable circuitry is to identify a first complete traversal between a first end of a track associated with the six-minute walk test and a second end of the track in response to a change of the strength of the signals received from the mobile beacon satisfying a change threshold.

Example 4 includes the system of example 1, wherein the programmable circuitry is to identify a local minimum or maximum signal strength in response to identifying a change in a signal strength slope between a positive slope and a negative slope in the strength of the signals received from the mobile beacon, and determine the local minimum or maximum signal strength corresponds to the subject turning around at a first end of a track associated with the six-minute walk test or a second end of the track in response to (a) a strength change following the local minimum or maximum signal strength satisfying a difference threshold and (b) the local minimum or maximum signal strength being within a threshold range of an average local minimum or maximum signal strength for the six-minute walk test.

Example 5 includes the system of example 4, wherein the programmable circuitry is to determine an estimated speed of the subject based on the signal strength slope, and determine the difference threshold based on the estimated speed.

Example 6 includes the system of example 4, wherein the programmable circuitry is to identify a first signal strength range associated with a complete traversal between the first end and the second end of the track based on a first local minimum signal strength and a first local maximum signal strength, identify a partially completed traversal in response to a second local maximum signal strength or a second local minimum signal strength not being within the threshold range of the average local minimum or maximum for the six-minute walk test, and determine a fraction of the track that the subject traversed during the partially completed traversal based on a ratio between the first signal strength range and a second signal strength range between the second local maximum signal strength and the second local minimum signal strength.

Example 7 includes the system of example 6, wherein the complete traversal is a first complete traversal of a plurality of complete traversals, and wherein the programmable circuitry is to determine a sum of a quantity of the plurality of complete traversals and the fraction of the track associated with the partially completed traversal, and multiply the sum by a predetermined distance of the track to determine the distance traveled by the subject during the six-minute walk test.

Example 8 includes the system of example 1, wherein the programmable circuitry is to identify a first quantity of rests reported by the subject, determine a second quantity of rests based on plateaus in the strength of signals received from the mobile beacon, and determine a confidence score associated with the determined distance based on the first quantity of rests and the second quantity of rests.

Example 9 includes a non-transitory machine readable storage medium comprising instructions to cause programmable circuitry to at least determine a distance traveled by a subject during a six-minute walk test based on signals received from a mobile beacon carried by the subject during the six-minute walk test.

Example 10 includes the non-transitory machine readable storage medium of example 9, wherein the instructions cause the programmable circuitry to determine a quantity of traversals that the subject completed between a first end of a track associated with the six-minute walk test and a second end of the track based on a rate of change of a strength of the signals received from the mobile beacon.

Example 11 includes the non-transitory machine readable storage medium of example 9, wherein the instructions cause the programmable circuitry to identify a first complete traversal between a first end of a track associated with the six-minute walk test and a second end of the track in response to a change of the signals received from the mobile beacon satisfying a change threshold.

Example 12 includes the non-transitory machine readable storage medium of example 9, wherein the instructions cause the programmable circuitry to identify a local minimum or maximum signal strength in response to identifying a change in a signal strength slope between a positive slope and a negative slope in the signals received from the mobile beacon, and determine the local minimum or maximum signal strength corresponds to the subject turning around at a first end of a track associated with the six-minute walk test or a second end of the track in response to (a) a strength change following the local minimum or maximum signal strength satisfying a difference threshold and (b) the local minimum or maximum signal strength being within a threshold range of an average local minimum or maximum signal strength for the six-minute walk test.

Example 13 includes the non-transitory machine readable storage medium of example 12, wherein the instructions cause the programmable circuitry to determine an estimated speed of the subject based on the signal strength slope, and determine the difference threshold based on the estimated speed.

Example 14 includes the non-transitory machine readable storage medium of example 12, wherein the instructions cause the programmable circuitry to identify a first signal strength range associated with a complete traversal between the first end and the second end of the track based on a first local minimum signal strength and a first local maximum signal strength, identify a partially completed traversal in response to a second local maximum signal strength or a second local minimum signal strength not being within the threshold range of the average local minimum or maximum for the six-minute walk test, and determine a fraction of the track that the subject traversed during the partially completed traversal based on a ratio between the first signal strength range and a second signal strength range between the second local maximum signal strength and the second local minimum signal strength.

Example 15 includes the non-transitory machine readable storage medium of example 14, wherein the complete traversal is a first complete traversal of a plurality of complete traversals, and wherein the instructions cause the programmable circuitry to determine a sum of a quantity of the plurality of complete traversals and the fraction of the track associated with the partially completed traversal, and multiply the sum by a predetermined distance of the track to determine the distance traveled by the subject during the six-minute walk test.

Example 16 includes the non-transitory machine readable storage medium of example 9, wherein the instructions cause the programmable circuitry to identify a first quantity of rests reported by the subject, determine a second quantity of rests based on plateaus in a strength of the signals received from the mobile beacon, and determine a confidence score associated with the determined distance based on the first quantity of rests and the second quantity of rests.

Example 17 includes a method comprising determining a distance traveled by a subject during a six-minute walk test based on signals received from a mobile beacon carried by the subject during the six-minute walk test.

Example 18 includes the method of example 17, further including identifying a local minimum or maximum signal strength in response to identifying a change in a signal strength slope between a positive slope and a negative slope in the signals received from the mobile beacon, and determining the local minimum or maximum signal strength corresponds to the subject turning around at a first end of a track associated with the six-minute walk test or a second end of the track in response to (a) a strength change following the local minimum or maximum signal strength satisfying a difference threshold and (b) the local minimum or maximum signal strength being within a threshold range of an average local minimum or maximum signal strength for the six-minute walk test.

Example 19 includes the method of example 18, further including determining an estimated speed of the subject based on the signal strength slope, and determining the difference threshold based on the estimated speed.

Example 20 includes the method of example 17, further including identifying a first quantity of rests reported by the subject, determining a second quantity of rests based on plateaus in a strength of the signals received from the mobile beacon, and determining a confidence score associated with the determined distance based on the first quantity of rests and the second quantity of rests.

Example 21 includes a system comprising a first stationary beacon to be positioned at a first end of a track to be traversed by the subject, a second stationary beacon to be positioned at a second end of the track opposite the first end, a mobile beacon to be carried by the subject during the six-minute walk test, memory, machine readable instructions, and programmable circuitry to at least one of instantiate or execute the machine readable instructions to determine a distance traveled by the subject during the six-minute walk test based on a strength of signals received from the mobile beacon.

Example 22 includes the system of example 21, wherein the programmable circuitry is to determine a quantity of traversals that the subject completed between the first stationary beacon and the second stationary beacon based on the strength of the signals received from the mobile beacon.

Example 23 includes the system of examples 21 or 22, wherein the programmable circuitry is to identify a local minimum or maximum signal strength in response to identifying a strength slope change between positive and negative slope in the strength of the signals received from the mobile beacon, and determine the local minimum or maximum corresponds to the subject turning around at the first end or the second end of the track in response to (a) a strength change following the local minimum or maximum satisfying a difference threshold and (b) the local minimum or maximum being within a threshold range of an average local minimum or maximum for the six-minute walk test.

Example 24 includes the system of example 23, wherein the programmable circuitry is to determine that the subject performed a complete traversal between the first end and the second end of the track when a consecutive local minimum and local maximum correspond to the subject turning around at the first end or the second end of the track.

Example 25 includes the system of example 24, wherein the programmable circuitry is to identify a signal strength range associated with a complete traversal between the first end and the second end of the track based on a first strength range between the consecutive local minimum and local maximum, identify a partially completed traversal in response to a local maximum or minimum not being within the threshold range of the average local minimum or maximum for the six-minute walk test, and determine a fraction of the track that the subject traversed during the partially completed traversal based on a ratio between the first strength range and a second strength range associated with a difference between the local maximum or minimum and a subsequent local minimum or maximum.

Example 26 includes the system of example 25, wherein the complete traversal is a first complete traversal of a plurality of complete traversals, and wherein the programmable circuitry is to determine a sum of a quantity of the plurality of complete traversals and the fraction of the track associated with the partially completed traversal, and multiply the sum by a predetermined distance of the track to determine the distance traveled by the subject during the six-minute walk test.

Example 27 includes the system of any of examples 21-26, wherein the programmable circuitry is to determine the subject was at the first end of the track when a local maximum in the strength was encountered.

The following claims are hereby incorporated into this Detailed Description by this reference. Although certain example systems, methods, apparatus, and articles of manufacture have been disclosed herein, the scope of coverage of this patent is not limited thereto. On the contrary, this patent covers all systems, methods, apparatus, and articles of manufacture fairly falling within the scope of the claims of this patent.

What is claimed is:

1. A system comprising:
a mobile beacon to be carried by a subject during a six-minute walk test;
interface circuitry;
machine readable instructions; and
programmable circuitry to at least one of instantiate or execute the machine readable instructions to determine a distance traveled by the subject during the six-minute walk test based on a strength of signals received from the mobile beacon.

2. The system of claim 1, wherein the programmable circuitry is to determine a quantity of traversals that the subject completed between a first end of a track associated with the six-minute walk test and a second end of the track based on a rate of change of the strength of the signals received from the mobile beacon.

3. The system of claim 1, wherein the programmable circuitry is to identify a first complete traversal between a first end of a track associated with the six-minute walk test and a second end of the track in response to a change of the strength of the signals received from the mobile beacon satisfying a change threshold.

4. The system of claim 1, wherein the programmable circuitry is to:
identify a local minimum or maximum signal strength in response to identifying a change in a signal strength slope between a positive slope and a negative slope in the strength of the signals received from the mobile beacon; and
determine the local minimum or maximum signal strength corresponds to the subject turning around at a first end of a track associated with the six-minute walk test or a second end of the track in response to (a) a strength change following the local minimum or maximum signal strength satisfying a difference threshold and (b) the local minimum or maximum signal strength being within a threshold range of an average local minimum or maximum signal strength for the six-minute walk test.

5. The system of claim 4, wherein the programmable circuitry is to:
determine an estimated speed of the subject based on the signal strength slope; and
determine the difference threshold based on the estimated speed.

6. The system of claim 4, wherein the programmable circuitry is to:
identify a first signal strength range associated with a complete traversal between the first end and the second end of the track based on a first local minimum signal strength and a first local maximum signal strength;
identify a partially completed traversal in response to a second local maximum signal strength or a second local minimum signal strength not being within the threshold range of the average local minimum or maximum for the six-minute walk test; and
determine a fraction of the track that the subject traversed during the partially completed traversal based on a ratio between the first signal strength range and a second signal strength range between the second local maximum signal strength and the second local minimum signal strength.

7. The system of claim 6, wherein the complete traversal is a first complete traversal of a plurality of complete traversals, and wherein the programmable circuitry is to:
determine a sum of a quantity of the plurality of complete traversals and the fraction of the track associated with the partially completed traversal; and
multiply the sum by a predetermined distance of the track to determine the distance traveled by the subject during the six-minute walk test.

8. The system of claim 1, wherein the programmable circuitry is to:
identify a first quantity of rests reported by the subject;
determine a second quantity of rests based on plateaus in the strength of signals received from the mobile beacon; and
determine a confidence score associated with the determined distance based on the first quantity of rests and the second quantity of rests.

9. A non-transitory machine readable storage medium comprising instructions to cause programmable circuitry to at least determine a distance traveled by a subject during a six-minute walk test based on signals received from a mobile beacon carried by the subject during the six-minute walk test.

10. The non-transitory machine readable storage medium of claim 9, wherein the instructions cause the programmable circuitry to determine a quantity of traversals that the subject completed between a first end of a track associated with the six-minute walk test and a second end of the track based on a rate of change of a strength of the signals received from the mobile beacon.

11. The non-transitory machine readable storage medium of claim 9, wherein the instructions cause the programmable circuitry to identify a first complete traversal between a first end of a track associated with the six-minute walk test and a second end of the track in response to a change of the signals received from the mobile beacon satisfying a change threshold.

12. The non-transitory machine readable storage medium of claim 9, wherein the instructions cause the programmable circuitry to:
identify a local minimum or maximum signal strength in response to identifying a change in a signal strength slope between a positive slope and a negative slope in the signals received from the mobile beacon; and
determine the local minimum or maximum signal strength corresponds to the subject turning around at a first end of a track associated with the six-minute walk test or a second end of the track in response to (a) a strength change following the local minimum or maximum signal strength satisfying a difference threshold and (b) the local minimum or maximum signal strength being within a threshold range of an average local minimum or maximum signal strength for the six-minute walk test.

13. The non-transitory machine readable storage medium of claim 12, wherein the instructions cause the programmable circuitry to:
determine an estimated speed of the subject based on the signal strength slope; and
determine the difference threshold based on the estimated speed.

14. The non-transitory machine readable storage medium of claim 12, wherein the instructions cause the programmable circuitry to:
identify a first signal strength range associated with a complete traversal between the first end and the second end of the track based on a first local minimum signal strength and a first local maximum signal strength;
identify a partially completed traversal in response to a second local maximum signal strength or a second local minimum signal strength not being within the threshold range of the average local minimum or maximum for the six-minute walk test; and
determine a fraction of the track that the subject traversed during the partially completed traversal based on a ratio between the first signal strength range and a second signal strength range between the second local maximum signal strength and the second local minimum signal strength.

15. The non-transitory machine readable storage medium of claim 14, wherein the complete traversal is a first complete traversal of a plurality of complete traversals, and wherein the instructions cause the programmable circuitry to:
determine a sum of a quantity of the plurality of complete traversals and the fraction of the track associated with the partially completed traversal; and
multiply the sum by a predetermined distance of the track to determine the distance traveled by the subject during the six-minute walk test.

16. The non-transitory machine readable storage medium of claim 9, wherein the instructions cause the programmable circuitry to:
identify a first quantity of rests reported by the subject;
determine a second quantity of rests based on plateaus in a strength of the signals received from the mobile beacon; and
determine a confidence score associated with the determined distance based on the first quantity of rests and the second quantity of rests.

17. A method comprising:
determining a distance traveled by a subject during a six-minute walk test based on signals received from a mobile beacon carried by the subject during the six-minute walk test.

18. The method of claim 17, further including:
identifying a local minimum or maximum signal strength in response to identifying a change in a signal strength slope between a positive slope and a negative slope in the signals received from the mobile beacon; and
determining the local minimum or maximum signal strength corresponds to the subject turning around at a first end of a track associated with the six-minute walk test or a second end of the track in response to (a) a strength change following the local minimum or maximum signal strength satisfying a difference threshold and (b) the local minimum or maximum signal strength being within a threshold range of an average local minimum or maximum signal strength for the six-minute walk test.

19. The method of claim 18, further including:
determining an estimated speed of the subject based on the signal strength slope; and
determining the difference threshold based on the estimated speed.

20. The method of claim 17, further including:
identifying a first quantity of rests reported by the subject;
determining a second quantity of rests based on plateaus in a strength of the signals received from the mobile beacon; and
determining a confidence score associated with the determined distance based on the first quantity of rests and the second quantity of rests.
